(19) United States
(12) Patent Application Publication
(10) Pub. No.: US 2007/0042050 A1
(43) Pub. Date: Feb. 22, 2007

(54) Nucleic acid vaccine compositions having a mammalian CD80/CD86 gene promoter driving antigen expression

(75) Inventor: Scott Umlauf, Madison, WI (US)

Correspondence Address: FOLEY AND LARDNER LLP;SUITE 500, 3000 K STREET NW, WASHINGTON, DC, 20007, US

(73) Assignee: Powderject Research Limited

(21) Appl. No.: 11/505,326

(22) Filed: Aug. 17, 2006

Related U.S. Application Data
(63) Continuation of application No. 10/748,124, filed on Dec. 31, 2003, now abandoned.
Application No. 10/748,124 is a continuation of application No. 09/705,022, filed on Nov. 1, 2000, now abandoned.
(60) Provisional application No. 60/163,195, filed on Nov. 3, 1999.

(51) Int. Cl. A61K 48/00; A61K 38/19; C07H 21/02; A61K 9/14

(52) U.S. Cl. 424/489; 514/44; 536/23.1; 424/85.1; 977/906

(57) ABSTRACT

Polynucleotides encoding at least one immunizing antigen whose expression is controlled by a promoter derived from a gene encoding a co-stimulatory molecule are provided. The polynucleotides may also encode adjuvants. Compositions comprising at least one immunizing agent and at least one cytokine that enhance dendritic cell stimulation and/or survival are also provided. Methods for eliciting an immune response against the immunizing agent are also provided. The method includes the steps of administering the polynucleotides and, optionally, co-administering an adjuvant.

### CROSS-REFERENCE TO RELATED PATENT APPLICATIONS

This application is a Continuation of U.S. application Ser. No. 10/748,124, filed Dec. 31, 2003, incorporated herein by reference in its entirety, which is a Continuation of U.S. application Ser. No. 09/705,022, filed Nov. 1, 2000, incorporated herein by reference in its entirety, which claims priority from Provisional Application U.S. Application 60/163,195, filed Nov. 3, 1999, incorporated herein by reference in its entirety.

### FIELD OF THE INVENTION

The present invention relates generally to vaccine compositions and methods of use thereof. More particularly, the invention pertains to polynucleotides encoding at least one immunizing antigen whose expression is controlled by a promoter derived from a co-stimulatory molecule. Methods of immunization using these polynucleotides are also provided. Also provided are compositions comprising at least one immunizing agent and at least one cytokine involved in maturation of antigen-presenting cells. Methods of eliciting an immune response using these compositions are also described.

### BACKGROUND OF THE INVENTION

Vaccines which induce a cell-mediated immune response are emerging as important strategies in combating parasites, autoimmune disorders, allergic diseases and cancers. Conventional vaccination strategies generally involve administration of either “live” or “dead” vaccines. Ertl et al. (1996) J. Immunol. 156:3579-3582. The so-called live vaccines include attenuated microbes and recombinant molecules based on a living vector. The dead vaccines include those based on killed whole pathogens, and subunit vaccines, e.g., soluble pathogen subunits or protein subunits. Live vaccines are generally successful in providing an effective immune response in immunized subjects; however, such vaccines can be dangerous in immunocompromised or pregnant subjects, can revert to pathogenic organisms, or can be contaminated with other pathogens. Hassett et al. (1996) Trends in Microbiol. 8:307-312. Dead vaccines avoid the safety problems associated with live vaccines; however such vaccines often fail to provide an appropriate and/or effective immune response in immunized subjects.

More recently, direct injection of plasmid DNA by intramuscular (Wolff et al. (1990) Science 247:1465:1468) or intradermal injection with a needle and syringe (Raz et al. (1994) PNAS USA 91:9519-9523) has been described. Another approach referred to as ballistic or particle-mediated DNA delivery employs a needless particle delivery device to administer DNA-coated microscopic gold beads directly into the cells of the epidermis. (Yang et al. (1990) PNAS USA 87:9568-9572). Thus, a number of delivery techniques can be used to deliver nucleic acids for immunizations, including particle-mediated techniques which deliver nucleic acid-coated microparticles into target tissue (see, e.g., co-owned U.S. Pat. No. 5,865,796, issued Feb. 2, 1999). Particle-mediated nucleic acid immunization techniques have been shown to elicit both humoral and cytotoxic T lymphocyte immune responses following epidermal delivery of nanogram quantities of DNA. Pertmer et al. (1995) Vaccine 13:1427-1430. Such particle-mediated delivery techniques have been compared to other types of nucleic acid inoculation, and found markedly superior. Fynan et al. (1995) Int. J. Immunopharmacology 17:79-83, Fynan et al. (1993) Proc. Natl. Acad. Sci. USA 90:11478-11482, and Raz et al. (1994) Proc. Natl. Acad. Sci. USA 91:9519-9523.

A novel transdermal drug delivery system that entails the use of a needleless syringe to deliver solid drug-containing particles in controlled doses into and through intact skin has also been described. In particular, commonly owned U.S. Pat. No. 5,630,796 to Bellhouse et al., describes a particle delivery device (e.g., a needleless syringe) that delivers pharmaceutical particles entrained in a supersonic gas flow. The particle delivery device is used for transdermal delivery of powdered drug compounds and compositions, for delivery of genetic material into living cells (e.g., gene therapy) and for the delivery of biopharmaceuticals to skin, muscle, blood or lymph. The device can also be used in conjunction with surgery to deliver drugs and biologics to organ surfaces, solid tumors and/or to surgical cavities (e.g., tumor beds or cavities after tumor resection). Pharmaceutical agents that can be suitably prepared in a substantially solid, particulate form can be safely and easily delivered using such a device.

One particular particle delivery device generally comprises an elongate tubular nozzle having a rupturable membrane initially closing the passage through the nozzle and arranged substantially adjacent to the upstream end of the nozzle. Particles of a therapeutic agent to be delivered are disposed adjacent to the rupturable membrane and are delivered using an energizing means which applies a gaseous pressure to the upstream side of the membrane sufficient to burst the membrane and produce a supersonic gas flow (containing the pharmaceutical particles) through the nozzle for delivery from the downstream end thereof. The particles can thus be delivered from the needleless syringe at delivery velocities of between Mach 1 and Mach 8 which are readily obtainable upon the bursting of the rupturable membrane.

Another particle delivery device configuration generally includes the same elements as described above, except that instead of having the pharmaceutical particles entrained within a supersonic gas flow, the downstream end of the nozzle is provided with a bistable diaphragm which is moveable between a resting “inverted” position (in which the diaphragm presents a concavity on the downstream face to contain the pharmaceutical particles) and an active “everted” position (in which the diaphragm is outwardly convex on the downstream face as a result of a supersonic shockwave having been applied to the upstream face of the diaphragm). In this manner, the pharmaceutical particles contained within the concavity of the diaphragm are expelled at a high initial velocity from the device for transdermal delivery thereof to a targeted skin or mucosal surface.

Transdermal delivery using the above-described device configurations is generally carried out with particles having an approximate size that generally ranges between 0.1 and 250 μm. Particles larger than about 250 μm can also be delivered from the device, with the upper limitation being the point at which the size of the particles would cause untoward damage to the skin cells. The actual distance which the delivered particles will penetrate depends upon particle size (e.g., the nominal particle diameter assuming a roughly spherical particle geometry), particle density, the initial velocity at which the particle impacts the skin surface, and the density and kinematic viscosity of the skin. Target particle densities for use in needleless particle injection generally range between about 0.1 and 25 g/cm³, and injection velocities generally range between about 150 and 3,000 m/sec.

### SUMMARY OF THE INVENTION

The present invention is directed to a polynucleotide comprising a first promoter derived from a gene encoding a co-stimulatory molecule and a first sequence encoding at least one antigen wherein the first sequence is operably linked to the first promoter. In particular embodiments, the promoter is derived from a CD80 (B7-1) gene or a CD86 (B7-2) gene.

In additional embodiments, the polynucleotide further comprises a second sequence encoding at least one cytokine operably linked to the first promoter or, alternatively, to a second promoter. The promoter may be a constituitive promoter.

In other embodiments, the invention is directed to a core carrier coated with a polynucleotide as described above, as well as to pharmaceutical compositions comprising the polynucleotide and a pharmaceutically acceptable excipient. The pharmaceutical compositions optionally further include a cytokine.

In still further embodiments, the invention is directed to a vaccine composition comprising (a) an expression vector comprising a polynucleotide encoding at least one antigen; and (b) a cytokine selected from the group consisting of CD40 ligand (CD40L), tumor-necrosis factor-related activation-induced cytokine (TRANCE) and Flt3 ligand (flt-3L).

In another embodiment, the invention is directed to a vaccine composition comprising (a) at least one peptide antigen; and (b) an expression vector comprising a polynucleotide encoding a cytokine selected from the group consisting of CD40 ligand (CD40L), tumor-necrosis factor-related activation-induced cytokine (TRANCE) and Flt3 ligand (flt-3L).

In yet another embodiment, the invention is directed to a vaccine composition comprising: (a) at least one peptide antigen; and (b) a cytokine selected from the group consisting of CD40 ligand (CD40L), tumor-necrosis factor-related activation-induced cytokine (TRANCE) and Flt3 ligand (flt-3L).

Methods for eliciting an immune response in a vertebrate subject comprising administering the vaccines above are also provided.

The various components of the above vaccine compositions may be coated onto a core carrier and used in methods for eliciting an immune response in a vertebrate subject. In this context, the method comprises administering the compositions to the subject using a particle-mediated delivery technique.

In another embodiment, the invention is directed to a method for eliciting an immune response in a vertebrate subject. The method comprises (a) providing a nucleotide sequence encoding an antigen operably linked to a promoter derived from a gene encoding a co-stimulatory molecule, the promoter capable of directing the expression of the antigen in the subject; and (b) administering the nucleotide sequence to the subject in an amount sufficient for the antigen to be expressed and elicit an immune response in the subject.

In a further embodiment, the invention is directed to a method for eliciting an immune response in a vertebrate subject. The method comprises (a) providing a particle coated with a nucleotide sequence encoding at least one antigen, the nucleotide sequence operably linked to a promoter derived from a gene encoding a co-stimulatory molecule, wherein the promoter is capable of driving expression of the antigen-encoding sequence in the subject; and (b) administering the particle to the subject using a particle-mediated delivery technique, whereby the antigen encoded by the nucleotide sequence is expressed in an amount sufficient to elicit an immune response.

In the methods above, the nucleotide sequence may further comprise a polynucleotide encoding at least one cytokine, such as a cytokine selected from the group consisting of CD40L, tumor-necrosis factor-related activation-induced cytokine (TRANCE) and Flt3 ligand (flt-3L).

These and other embodiments of the present invention will readily occur to those of ordinary skill in the art in view of the disclosure herein.

### BRIEF DESCRIPTION OF THE DRAWINGS

FIG. 1 is a schematic representation of the CD80 promoter-driven expression vector p5020. This plasmid vector was constructed from pWRG7128, a mammalian expression vector based on a pUC19 backbone. The CMV promoter was removed and replaced with a 254 base pair PCR fragment obtained by amplification of the mouse CD80 promoter, obtained from Life Technologies, Gibco BRL. The plasmid also contains regulatory elements, and the polyA signal of bovine growth hormone, operably linked to the full length cDNA encoding the Hepatitis B surface antigen.

FIG. 2 is a schematic representation of the CD80 promoter-driven expression vector p5021. This plasmid vector was constructed from pWRG7128, a mammalian expression vector based on a pUC19 backbone. The CMV promoter was removed and replaced with a 489 base pair PCR fragment obtained by amplification of the mouse CD80 promoter, obtained from Life Technologies, Gibco BRL. The plasmid also contains regulatory elements, and the polyA signal of bovine growth hormone, operably linked to the full length cDNA encoding the Hepatitis B surface antigen.

FIG. 3 is a schematic representation of the CD80 promoter-driven expression vector p5022. This plasmid vector was constructed from pWRG7128, a mammalian expression vector based on a pUC19 backbone. The CMV promoter was removed and replaced with a 3123 base pair PCR fragment obtained by amplification of the mouse CD80 promoter, obtained from Life Technologies, Gibco BRL. The plasmid also contains regulatory elements, and the polyA signal of bovine growth hormone, operably linked to the full length cDNA encoding the Hepatitis B surface antigen.

FIG. 4 is a schematic representation of the CD80 promoter-driven expression vector p5023. This plasmid vector was constructed from pWRG7128, a mammalian expression vector based on a pUC19 backbone. The CMV promoter was removed and replaced with a 3357 base pair PCR fragment obtained by amplification of the mouse CD80 promoter, obtained from Life Technologies, Gibco BRL. The plasmid also contains regulatory elements, and the polyA signal of bovine growth hormone, operably linked to the full length cDNA encoding the Hepatitis B surface antigen.

FIG. 5 is a schematic representation of the CD80 promoter-driven expression vector p5024. This plasmid vector was constructed from pWRG7128, a mammalian expression vector based on a pUC19 backbone. The CMV promoter was removed and replaced with a 578 base pair PCR fragment obtained by amplification of the human CD80 promoter, obtained from Life Technologies, Gibco BRL. The plasmid also contains regulatory elements, and the polyA signal of bovine growth hormone, operably linked to the full length cDNA encoding the Hepatitis B surface antigen.

FIG. 6 is a schematic representation of the CD80 promoter-driven expression vector p5025. This plasmid vector was constructed from pWRG7128, a mammalian expression vector based on a pUC19 backbone. The CMV promoter was removed and replaced with a 202 base pair PCR fragment obtained by amplification of the human CD80 promoter, obtained from Life Technologies, Gibco BRL. The plasmid also contains regulatory elements, and the polyA signal of bovine growth hormone, operably linked to the full length cDNA encoding the Hepatitis B surface antigen.

FIG. 7 is a schematic representation of the CD80 promoter-driven expression vector p5026. This plasmid vector was constructed from pWRG7128, a mammalian expression vector based on a pUC19 backbone. The CMV promoter was removed and replaced with a 294 base pair PCR fragment obtained by amplification of the human CD80 promoter, obtained from Life Technologies, Gibco BRL. The plasmid also contains regulatory elements, and the polyA signal of bovine growth hormone, operably linked to the full length cDNA encoding the Hepatitis B surface antigen.

FIG. 8 is a graph depicting cytotoxic T cell (CTL) responses elicited in mice immunized with plasmids encoding hepatitis B surface antigen (HBsAg) under the control of CMV promoter or CD80 promoters.

FIG. 9 is a histogram depicting anti-hepatitis B core antigen specific IL-4 production in splenocytes from mice immunized with plasmids encoding hepatitis B core/surface antigens and a TRANCE cytokine adjuvant.

FIG. 10 is a graph depicting anti-hepatitis B core antigen specific antibody production in mice immunized with plasmids encoding hepatitis B core/surface antigens and a TRANCE cytokine adjuvant.

### MODES FOR CARRYING OUT THE INVENTION

Before describing the present invention in detail, it is to be understood that this invention is not limited to particular antigens or to antigen-coding nucleotide sequences. It is also to be understood that different applications of the disclosed methods may be tailored to the specific needs in the art. It is also to be understood that the terminology used herein is for the purpose of describing particular embodiments of the invention only, and is not intended to be limiting.

All publications, patents and patent applications cited herein, whether supra or infra are hereby incorporated by reference in their entirety.

It must be noted that, as used in this specification and the appended claims, the singular forms “a”, “an”, and “the” include plural referents unless the content clearly dictates otherwise. Thus, for example, reference to “an antigen” includes a mixture of two or more such agents, reference to “a particle” includes reference to mixtures of two or more particles, reference to “a recipient cell” includes two or more such cells, and the like.

### Definitions

Unless defined otherwise, all technical and scientific terms used herein have the same meaning as commonly understood by one of ordinary skill in the art to which the invention pertains. The following terms are intended to be defined as indicated below.

The term “vaccine composition” intends any pharmaceutical composition containing an antigen (e.g., polynucleotide encoding an antigen), which composition can be used to prevent or treat a disease or condition in a subject. The term thus encompasses both subunit vaccines, i.e., vaccine compositions containing antigens which are separate and discrete from a whole organism with which the antigen is associated in nature, as well as compositions containing whole killed, attenuated or inactivated bacteria, viruses, parasites or other microbes.

The term “transdermal” delivery intends intradermal (e.g., into the dermis or epidermis), transdermal (e.g., “percutaneous”) and transmucosal administration, i.e., delivery by passage of an agent into or through skin or mucosal tissue. See, e.g., Transdermal Drug Delivery: Developmental Issues and Research Initiatives, Hadgraft and Guy (eds.), Marcel Dekker, Inc., (1989); Controlled Drug Delivery. Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Thus, the term encompasses delivery of particles from a particle delivery device (e.g., needleless syringe) as described in U.S. Pat. No. 5,630,796, as well as particle-mediated delivery of coated core carriers as described in U.S. Pat. No. 5,865,796.

By “core carrier” is meant a carrier particle on which a nucleic acid (e.g., DNA) is coated in order to impart a defined particle size as well as a sufficiently high density to achieve the momentum required for cell membrane penetration, such that the DNA can be delivered using particle-mediated delivery techniques, for example those described in U.S. Pat. No. 5,100,792. Core carriers typically include materials such as tungsten, gold, platinum, ferrite, polystyrene and latex. See e.g., Particle Bombardment Technology for Gene Transfer, (1994) Yang, N. ed., Oxford University Press, New York, N.Y. pages 10-11.

By “particle delivery device,” or “needleless syringe,” is meant an instrument which delivers a particulate composition transdermally, without a conventional needle that pierces the skin. Particle delivery devices for use with the present invention are discussed throughout this document.

By “antigen” is meant a molecule which contains one or more epitopes that will stimulate a host's immune system to make a cellular antigen-specific immune response, or a humoral antibody response. Thus, antigens include proteins, polypeptides, antigenic protein fragments, oligosaccharides, polysaccharides, and the like. Furthermore, the antigen can be derived from any known virus, bacterium, parasite, plants, protozoans, or fungus, and can be a whole organism. The term also includes tumor antigens. Similarly, an oligonucleotide or polynucleotide which expresses an antigen, such as in DNA immunization applications, is also included in the definition of antigen. Synthetic antigens are also included, for example, polyepitopes, flanking epitopes, and other recombinant or synthetically derived antigens (Bergmann et al. (1993) Eur. J. Immunol. 23:2777-2781; Bergmann et al. (1996) J. Immunol. 157:3242-3749; Suhrbier, A. (1997) Immunol. and Cell Biol. 75:402-408; Gardner et al. (1998) 12th World AIDS Conference, Geneva, Switzerland, Jun. 28-Jul. 3, 1998).

The term “peptide” is used in it broadest sense to refer to a compound of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is typically called a polypeptide or a protein

“T cell epitopes” are generally those features of a peptide structure capable of inducing a T cell response. In this regard, it is accepted in the art that T cell epitopes comprise linear peptide determinants that assume extended conformations within the peptide-binding cleft of MHC molecules, (Unanue et al. (1987) Science 29,:551-557). As used herein, a T cell epitope is generally a peptide having about 8-15, preferably 5-10 or more amino acid residues.

The term “antigen presenting cell” or “APC” as used herein, intends any cell which presents on its surface an antigen in association with a major histocompatibility complex molecule, or portion thereof, or, alternatively, one or more non-classical MHC molecules, or a portion thereof. Examples of suitable APCs are discussed in detail below and include, but are not limited to, whole cells such as Langerhans cells, macrophages, dendritic cells, B cells, hybrid APCs, and foster antigen presenting cells.

Dendritic cells (DCs) and Langerhans cells are potent antigen-presenting cells. DCs are minor constituents of various immune organs, for example, constituting around 1% of epidermal cell suspensions (Schuler et al. (1985) J. Exp. Med. 161:526; and Romani et al. (1989) J. Invest. Dermatol. 93:600). Despite their relative scarcity, these cells have been shown to provide all the signals required for T cell activation and proliferation. The requisite signals can be categorized into two types. The first type, which gives specificity to the immune response, is mediated through interaction between the T-cell receptor/CD3 (“TCR/CD3”) complex and an antigenic peptide presented by a major histocompatibility complex (“MHC”) class I or II protein on the surface of APCs. This interaction is necessary, but not sufficient, for T cell activation to occur. In fact, without the second type of signal, the first type of signal can result in T cell anergy (e.g., where T-cells are insensitive to additional signals). The second type of signal, called a co-stimulatory signal, is neither antigen-specific nor MHC-restricted, and can lead to a full proliferation response of T cells and induction of T cell effector functions in the presence of the first type of signal. Thus, as discussed above, research accumulated over the past several years has demonstrated convincingly that resting T cells require at least two signals for induction of cytokine gene expression and proliferation (Schwartz R. H. (1990) Science 248:1349-1356; Jenkins M. K. (1992) Immunol. Today 13:69-73). One signal, the one that confers specificity, can be produced by interaction of the TCR/CD3 complex with an appropriate MIHC/peptide complex. The second signal is not antigen specific and is termed the “co-stimulatory” signal.

“Co-stimulatory molecules” act as receptor-ligand pairs expressed on the surface of antigen presenting cells and T cells. The term encompasses any single molecule or combination of molecules which, when acting together with a peptide/MHC complex bound by a TCR on the surface of a T cell, provides a co-stimulatory effect which achieves activation of the T cell that binds the peptide.

Several molecules have been shown to enhance co-stimulatory activity. These are CD80 (i.e., B7-1), CD86 (i.e., B7-2/B70) (Schwartz R. H. (1992) Cell 71:1065-1068), heat stable antigen (HSA) (Liu Y. et al. (1992) J. Exp. Med. 175:437-445), chondroitin sulfate-modified MHC invariant chain (Naujokas M. F., et al. (1993) Cell 74:257-268), intracellular adhesion molecule 1 (ICAM-1) (Van Seventer, G. A. (1990) J. Immunol. 144:4579-4586). These molecules each appear to accomplish co-stimulation by interacting with their cognate ligands on the T cells. Co-stimulatory molecules mediate signal(s) which are necessary, under normal physiological conditions, to achieve full activation of nave T cells. One exemplary receptor-ligand pair are the CD80 and CD86 co-stimulatory molecule on the surface of APCs and their counter-receptors, CD28 and CTLA-4 on T cells (Ellis et al. (1996) J. Immunol. 56:2700-2709; Freeman et al. (1993) Science 262:909-911; Nabavi et al. (1992) Nature 360:266-268). Other important co-stimulatory molecules are CD40 and CD54. The term thus encompasses CD80, CD86, or other co-stimulatory molecule(s) on an antigen-presenting matrix such as an APC, as well as fragments of the co-stimulatory molecule(s) (alone, complexed with another molecule(s), or as part of a fusion protein) which binds to a cognate ligand and results in activation of the T cell when the TCR on the surface of the T cell specifically binds the peptide. Many of the sequences of the genes encoding co-stimulatory molecules and their promoter regions are known in the art. Other promoters or fragments thereof can also be determined by methods known in the art and described herein.

As used herein the term “adjuvant” refers to any material that enhances the action of a drug, antigen, polynucleotide, vector or the like. Thus, one example of an adjuvant is a “cytokine.” As used herein, the term “cytokine” refers to any one of the numerous factors that exert a variety of effects on cells, for example, inducing growth, proliferation or maturation. Certain cytokines, for example TRANCE, flt-3L, and CD40L, enhance the immunostimulatory capacity of APCs. Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1α), interleukin-11 (IL-11), MIP-1α, leukemia inhibitory factor (LIF), c-kit ligand, thromboproietin (TPO), CD40 ligand (CD40L), tumor necrosis factor-related activation-induced cytokine (TRANCE) and flt3 ligand (flt-3L). Cytokines are commercially available from several vendors such as, for example, Genzyme (Framingham, Mass.), Genentech (South San Francisco, Calif.), Amgen (Thousand Oaks, Calif.) R&D Systems and Immunex (Seattle, Wash.). The sequence of many of these molecules are also available, for example, from the GenBank database. It is intended, although not always explicitly stated, that molecules having similar biological activity as wild-type or purified cytokines (e.g., recombinantly produced or mutants thereof) and nucleic acid encoding these molecules are intended to be used within the spirit and scope of the invention.

A composition which contains a selected antigen and an adjuvant, or a vaccine composition which is co-administered with an adjuvant, displays “enhanced immunogenicity” when it possesses a greater capacity to elicit an immune response than the immune response elicited by an equivalent amount of the antigen administered without the adjuvant. Thus, a vaccine composition may display “enhanced immunogenicity” because the antigen is more strongly immunogenic or because a lower dose or fewer doses of antigen are necessary to achieve an immune response in the subject to which the antigen is administered. Such enhanced immunogenicity can be determined by administering the adjuvant composition and antigen controls to animals and comparing antibody titers and/or cellular-mediated immunity between the two using standard assays such as radioimmunoassay, ELISAs, CTL assays, and the like, well known in the art.

The terms “nucleic acid molecule” and “polynucleotide” are used interchangeably and refer to a polymeric form of nucleotides of any length, either deoxyribonucleotides or ribonucleotides, or analogs thereof. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

A polynucleotide is typically composed of a specific sequence of four nucleotide bases: adenine (A); cytosine (C); guanine (G); and thymine (T) (uracil (U) is substituted for thymine (T) when the polynucleotide is RNA). Thus, the term polynucleotide sequence is the alphabetical representation of a polynucleotide molecule. This alphabetical representation can be input into databases in a computer having a central processing unit and used for bioinformatics applications such as functional genomics and homology searching.

A “gene” as used in the context of the present invention is a sequence of nucleotides in a genetic nucleic acid (chromosome, plasmid, etc.) with which a genetic function is associated. A gene is a hereditary unit, for example of an organism, comprising a polynucleotide sequence (e.g., a DNA sequence for mammals) that occupies a specific physical location (a “gene locus” or “genetic locus”) within the genome of an organism. A gene can encode an expressed product, such as a polypeptide or a polynucleotide (e.g., tRNA). Alternatively, a gene may define a genomic location for a particular event/function, such as the binding of proteins and or nucleic acids (e.g., phage attachment sites), wherein the gene does not encode an expressed product. Typically, a gene includes coding sequences, such as polypeptide encoding sequences, and non-coding sequences, such as promoter sequences, poly-adenlyation sequences, transcriptional regulatory sequences (e.g., enhancer sequences). Many eucaryotic genes have “exons” (coding sequences) interrupted by “introns” (non-coding sequences). In certain cases, a gene may share sequences with another gene(s) (e.g., overlapping genes).

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence. Transcription and translation of coding sequences are typically regulated by “control elements,” including, but not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

A “promoter” is a nucleotide sequence which initiates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. In addition, such promoters can also have tissue specificity, for example, the CD80 promoter is only inducible in certain immune cells, and the myoD promoter is only inducible in muscle cells. It is intended that the term “promoter” or “control element” includes full-length promoter regions and functional (e.g., controls transcription or translation) segments of these regions. A promoter is “derived from” a gene encoding a co-stimulatory molecule if it has the same or substantially the same basepair sequence as a region of the promoter region of the co-stimulatory molecule, complements thereof, or if it displays sequence identity as described below.

A “vector” is capable of transferring gene sequences to target cells (e.g., viral vectors, non-viral vectors, particulate carriers, and liposomes). Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as viral vectors.

An “isolated polynucleotide” molecule is a nucleic acid molecule separate and discrete from the whole organism with which the molecule is found in nature; or a nucleic acid molecule devoid, in whole or part, of sequences normally associated with it in nature; or a sequence, as it exists in nature, but having heterologous sequences (as defined below) in association therewith.

“Operably linked” refers to an arrangement of elements wherein the components so described are configured so as to perform their usual function. Thus, a given promoter that is operably linked to a coding sequence (e.g., an antigen or interest) is capable of effecting the expression of the coding sequence when the regulatory proteins and proper enzymes are present. In some instances, certain control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

“Recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide.

Techniques for determining nucleic acid and amino acid “sequence identity” also are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of t,.vio sequences, whether nucleic acid or amino acid sequences, is the number of exact matches between two aligned sequences divided by the length of the shorter sequences and multiplied by 100. An appropriate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://wvw.ncbi.nlm.gov/cgi-bin/BLAST.

Alternatively, homology can be determined by hybridization of polynucleotides under conditions which from stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85%, preferably at least about 90%, and most preferably at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. For example, stringent hybridization conditions can include 50% formamide, 5× Denhardt's Solution, 5×SSC, 0.1% SDS and 100 μg/ml denatured salmon sperm DNA and the washing conditions can include 2×SSC, 0.1% SDS at 37° C. followed by 1×SSC, 0.1% SDS at 68° C. Defining appropriate hybridization conditions is within the skill of the art. See, e.g., Sambrook et al., supra; DNA Cloning, supra; Nucleic Acid Hybridization, supra.

As used herein, the term “treatment” includes any of following: the prevention of infection or reinfection; the reduction or elimination of symptoms; and the reduction or complete elimination of a pathogen. Treatment may be effected prophylactically (prior to infection) or therapeutically (following infection). An “effective amount” is an amount sufficient to effect beneficial or desired results. An effective amount can be administered in one or more administrations, applications of dosages. The term “co-administering” or “co-administration” refers to administration of at least two substances. Co-administration can be achieved by administering the substances concurrently or at different times. In addition, co-administration includes delivery using one or more delivery means.

By suitable immune response, it is meant that the methods of the invention can bring about in an immunized subject an immune response characterized by the production of B and/or T lymphocytes specific for a viral antigen, wherein the immune response can protect the subject against subsequent infection with homologous or heterologous viral strains, reduce viral burden and/or shedding during an infection, bring about resolution of infection in a shorter amount of time relative to a non-immunized subject, or prevent or reduce clinical manifestation of disease symptoms.

By “vertebrate subject” is meant any member of the subphylum cordata, particularly mammals, including, without limitation, humans and other primates. The term does not denote a particular age. Thus, both adult and newborn individuals are intended to be covered.

### General Overview of the Invention

Before describing the present invention in detail, it is to be understood that this invention is not limited to particular formulations or process parameters (as such may, of course, vary). It is also to be understood that the terminology used herein is for the purpose of describing particular embodiments of the invention only, and is not intended to be limiting.

DNA-vaccines generally consist of a plasmid that encodes a relevant antigen for de novo synthesis by cells present in a targeted tissue. Viral promoters, e.g., the promoter from Cytomegalovirus (CMV), are generally used in DNA-vaccine plasmid constructs to drive antigen expression. Delivery of these DNA-vaccine plasmids, both in “naked” form and attached to particles, has been shown to elicit both humoral and cell-mediated immune responses. (See, e.g., Wang et al. (1993) Proc. Natl. Acad. Sci. USA 90:4156-4160; Tang et al. (1992) Nature 356:152-154; Fynan, supra).

It is known that in order to provoke a specific CTL (cytotoxic T-cell) response, an antigen must be presented to T cells. This is accomplished via antigen presenting cells (APCs), a class of cells which includes dendritic cells (DCs), Langerhanis cells, monocytes, macrophages, and B cells. DCs were first described as the morphologically distinct Langerhans cells in the epidermis of the skin (as reviewed by Bancheraeau et al. (1996) Nature 392:245-252) and have since been shown to be the most efficient APC for the activation of nave T cells. Lanzavecohia A. (1993) Science 260:937-944 and Bancheraeau et al. (1998), supra. The antigens encoded by injected DNA-vaccines are processed into peptides and presented to T-cells by dendritic cells. It has also been shown that intraepidermally delivered DNA-vaccines target Langerhans cells. The targeted Langerhans cells express the DNA-encoded antigen and migrate out of the epidermis to the draining lymph nodes where they process and present the DNA-vaccine encoded antigen(s) to T-lymphocytes. Thus, APCs play an essential role in the induction of an effective immune response to DNA-vaccines.

APCs exposed to antigens process the antigens into small fragments, known as epitopes, which are then associated with the major histocompatibility complex (MHC) Class I for presentation to CD8+ T-lymphocytes and with MHC Class II for presentation to CD4+ T-lymphocytes. However, certain co-stimulatory molecules, for example CD80 and CD86 (also known as B7-1 and B7-2, respectively), are also required for antigen presentation. Thus, effective activation of T-lymphocytes requires two signals at the cell surface interface of the APC and the target T-cell. It is now known that the first activation signal is provided by binding of the T-cell receptor (TCR) to the antigen-MHC complex and second activation signal is provided by engagement of the CD80/CD86 co-stimulatory molecules on the APC with the CD28 receptor on the T-lymphocytes. After maturation, APCs become sensitive to apoptosis, thus limiting their natural stimulatory capacity.

The CD80/CD86 co-stimulatory molecules required for successful antigen presentation are not constitutively expressed by APCs. Rather, upon activation, e.g., in response to an infectious pathogen, CD80/CD86 expression on the surface of APCs is rapidly up-regulated. Signals for inducing CD80/CD86 expression by APCs can be provided by cytokines released by epithelial and lymphoid cells in an inflamed tissue site infected with a pathogen. Furthermore, certain cytokines secreted by activated T-lymphocytes, e.g., IFNγ, can both induce and maintain the expression of CD80/CD86 by APCs. To date, two members of the TNF family, CD40 ligand (CD40L or CD154), and TNF-related Activation-induced Cytokine (TRANCE), and a factor known as Flt3 ligand (Flt3L) have been implicated in APC maturation see, e.g., Gurunathan et al. (1998) J. Immunol., 161:4563-4571; Pulendran et al. (1998) J. Exp. Med., 188:2075-2082) and Wong et al. (1999) J. Immunol 162:2251-2258. TRANCE has also been shown to prolong the life-span of mature DC. (Josien et al. (1999) J. Immun. 162:2562-2568; Wong et al. (1997) J. Exp. Med., 186:2075-2080). Co-administration of CD40L and tumor specific antigens has been shown to result in production of IgG1 antibodies, reflecting a Th2-type immune response (see, Wong et al. (1999), supra).

Recently, methods have been described to enhance the T-cell response of a subject. These methods entail administering nucleotides encoding, under the same transcriptional regulatory element, an immunizing antigen and full-length co-stimulatory molecule. (See, e.g., U.S. Pat. No. 5,738,852 and International Publication WO 97/32987, published Sep. 12, 1997.) It is useful to note that these studies exemplify and describe expression of an immunizing agent and a co-stimulatory molecule under the control of a constitutive promoter, e.g., a CMV promoter.

There remains a need in the art for inducible, APC-targeted DNA vaccines and methods which effectively enhance maturation and the stimulatory lifespan of the targeted APCs, for example by targeting specific cells or being active only in specific cells. The invention described herein achieves this goal, for example by operably linking a polynucleotide encoding an immunizing agent to a promoter sequence derived from a gene encoding a co-stimulatory molecule, and/or by co-administering the immunizing agent with one or more cytokines that enhance the stimulatory lifespan of APCs.

More particularly, the present invention provides novel polynucleotides which are particularly useful as vaccines. Typically, the polynucleotides are carried on vectors, for instance plasmids, which contain suitable regulatory elements. In one embodiment, the polynucleotides of the present invention comprise a sequence encoding at least one selected antigen. Expression of the antigen(s) is controlled by a transcriptional regulatory element (e.g., promoter) derived from a gene encoding a co-stimulatory molecule, for example CD80 or CD86. Without being bound by a particular theory, it appears that using a promoter element derived from a co-stimulatory molecule takes advantage of the APC's normal up-regulation of these promoters upon activation and during antigen presentation. Thus, using the polynucleotides described herein enhances antigen expression, processing and presentation in APCs as compared to using polynucleotides driven by, for example, constitutive promoters.

The invention also includes compositions wherein an expression vector comprising a co-stimulatory molecule promoter and a sequence encoding an antigen further comprises an adjuvant, for example a cytokine. Preferably, the cytokine enhances the immune response, for example, by enhancing the immunostimulatory capacity of the APCs, increasing expression of co-stimulatory ligands on the surface of the APCs, stabilizing antigen/MHC complexes and/or inhibiting apoptosis of the APCs. Methods of co-administering polynucleotides carrying antigens operably linked to a co-stimulatory molecule promoter along with adjuvants are also included. The selected adjuvants may be given in the form of polynucleotides under suitable regulatory control or as polypeptides (e.g., recombinantly produced polypeptides). When administered as nucleotides, the cytokine-encoding sequence and antigen-encoding sequence may be carried on the same vector or on different vectors. Thus, the cytokine-encoding sequence may be under the control of a promoter derived from a co-stimulatory molecule or, alternatively, a different promoter (e.g., a constitutive promoter). Furthermore, the cytokine-encoding sequence may be located either 3′ or 5′ to the antigen-encoding sequence.

The invention further includes vaccine compositions comprising antigens in combination with at least one cytolkine that enhances stimulation or survival of dendritic cells. As described above, such cutokines (e.g., TRANCE, flt3, CD40L) may increase expression of co-stimulatory molecules on the surface of APCs, stabalize the antigen/MHC complex or prevent their apoptisis, thereby increasing the stimulatory lifespan of APCs. In particular, compositions comprising polynucleotides encoding at least one antigen and a peptide cytokine, particularly a cytokine such as TRANCE, flt-3L or CD40L, are described. Another composition includes those comprising at least one antigen (e.g., peptide) and at least one cytokine (e.g., TRANCE, flt-3L and/or CD40L). Yet another composition comprises at least one antigen (e.g., peptide) and at least one polynucleotide encoding a cytokine (e.g., TRANCE, flt-3L and/or CD40L). It is to be understood that more than one antigen can be used in combination with one or more cytokines.

The polynucleotides of the present invention may be introduced into cells in vitro or in vivo, for example by transfection or by coating the polynucleotides onto particles and administering the coated particles to the cells. Alternatively, the polynucleotides and/or peptides may be provided in a particulate (e.g., powder) form, discussed more fully below and in the disclosure of International Publication Numbers WO 97/48485 and WO 98/10750, which are incorporated by reference herein.

Thus, the invention includes methods for eliciting an immune response, preferably a CTL response, in a vertebrate subject by administering a polynucleotide encoding at least one selected antigen, where the antigen-encoding sequence is operably linked to a regulatory element of a co-stimulatory molecule. Also provided are methods for eliciting an immune response in a vertebrate subject by co-administering a selected antigen with a cytokine such as TRANCE, flt-3L or CD40L.

Antigens

The compositions and methods described herein are useful in eliciting an immune response against a wide variety of antigens for the treatment and/or prevention of a number of conditions including, but not limited to, cancer, allergies, toxicity and infection by pathogens such as viruses, bacteria, fungi, and other pathogenic organisms.

Suitable viral antigens for use in the present compositions and methods include, but are not limited to, those obtained or derived from the hepatitis family of viruses, including hepatitis A virus (HAV), hepatitis B virus (HBV), hepatitis C virus (HCV), the delta hepatitis virus (HDV), hepatitis E virus (HEV) and hepatitis G virus (HGV). See, e.g., International Publication Nos. WO 89/04669; WO 90/11089; and WO 90/14436. The HCV genome encodes several viral proteins, including E1 and E2. See, e.g., Houghton et al. (1991) Hepatology 14:381-388. Nucleic acid molecules containing sequences encoding these proteins, as well as antigenic fragments thereof, will find use in the present methods. Similarly, the coding sequence for the 8-antigen from HDV is known (see, e.g., U.S. Pat. No. 5,378,814).

In like manner, a wide variety of proteins from the herpesvirus family can be used as antigens in the present invention, including proteins derived from herpes simplex virus (HSV) types 1 and 2, such as HSV-1 and HSV-2glycoproteins gB, gD and gH; antigens from varicella zoster virus (VZV), Epstein-Barr virus (EBV) and cytomegalovirus (CMV) including CMV gB and gH; and antigens from other human herpesviruses such as HHV6 and HHV7. (See, e.g. Chee et al. (1990) Cytomegaloviruses (J. K. McDougall, ed., Springer Verlag, pp. 125-169; McGeoch et al. (1988) J. Gen. Virol. 69: 1531-1574; U.S. Pat. No. 5,171,568; Baer et al. (1984) Nature 310: 207-211; and Davison et al. (1986) J. Gen. Virol. 67: 1759-1816.)

Human immunodeficiency virus (HIV) antigens, such asgp120 molecules for a multitude of HIV-1 and HIV-2 isolates, including members of the various genetic subtypes of HIV, are known and reported (see, e.g., Myers et al., Los Alamos Database, Los Alamos National Laboratory, Los Alamos, N. Mex. (1992); and Modrow et al. (1987) J. Virol. 61: 570-578) and antigen-containing nucleic acid sequences derived or obtained from any of these isolates will find use in the present invention. Furthermore, other immunogenic proteins derived or obtained from any of the various HIV isolates will find use herein, including sequences encoding one or more of the various envelope proteins such as gp 160 and gp41, gag antigens such as p24gag and p55gag, as well as proteins derived from the pol, env, tat, vif, rev, nef, vpr, vpu and LTR regions of HIV.

Antigens derived or obtained from other viruses will also find use herein, such as without limitation, antigens from members of the families Picornaviridae (e.g., polioviruses, rhinoviruses, etc.); Caliciviridae; Togaviridae (e.g., rubella virus, dengue virus, etc.); Flaviviridae; Coronaviridae; Reoviridae (e.g., rotavirus, etc.); Bimaviridae; Rhabodoviridae (e.g., rabies virus, etc.); Orthomyxoviridae (e.g., influenza virus types A, B and C, etc.); Filoviridae; Paramyxoviridae (e.g., mumps virus, measles virus, respiratory syncytial virus, parainfluenza virus, etc.); Bunyaviridae; Arenaviridae; Retroviradae (e.g., HTLV-I; HTLV-II; HIV-1 (also known as HTLV-III, LAV, ARV, hTLR, etc.)), including but not limited to antigens from the isolates HIV_{IIIb}, HIV_{SF2}, HIV_{LAV}, HIV_{LAI}, HIV_{MN}); HIV-1_{CM235}, HIV-1_{US4}; HIV-2, among others; simian immunodeficiency virus (SIV); Papillomavirus, the tick-boume encephalitis viruses; and the like. See, e.g. Virology, 3rd Edition (W. K. Joklik ed. 1988); Fundamental Virology, 2nd Edition (B. N. Fields and D. M. Knipe, eds. 1991), for a description of these and other viruses.

In some contexts, it may be preferable that a selected antigen is obtained or derived from a viral pathogen that typically enters the body via a mucosal surface and is known to cause or is associated with human disease, such as, but not limited to, HIV (AIDS), influenza viruses (Flu), herpes simplex viruses (genital infection, cold sores, STDs), rotaviruses (diarrhea), parainfluenza viruses (respiratory infections), poliovirus (poliomyelitis), respiratory syncytial virus (respiratory infections), measles and mumps viruses (measles, mumps), rubella virus (rubella), and rhinoviruses (common cold).

Suitable bacterial and parasitic antigens can be obtained or derived from known causative agents responsible for diseases including, but not limited to, Diptheria, Pertussis, Tetanus, Tuberculosis, Bacterial or Fungal Pneumonia, Otitis Media, Gonnorhea, Cholera, Typhoid, Meningitis, Mononucleosis, Plague, Shigellosis or Salmonellosis, Legionaire's Disease, Lyme Disease, Leprosy, Malaria, Hookworm, Onchocerciasis, Schistosomiasis, Trypamasomialsis, Lesmaniasis, Giardia, Amoebiasis, Filariasis, Borelia, and Trichinosis. Still further antigens can be obtained or derived from unconventional pathogens such as the causative agents of kuru, Creutzfeldt-Jakob disease (CJD), scrapie, transmissible mink encephalopathy, and chronic wasting diseases, or from proteinaceous infectious particles such as prions that are associated with mad cow disease.

Specific pathogens from which antigens can be derived include M. tuberculosis, Chlamydia, N. gonorrhoeae, Shigella, Salmonella, Vibrio Cholera, Treponema pallidua, Pseudomonas, Bordetella pertussis, Brucella, Franciscella tulorensis, Helicobacter pylori, Leptospria interrogaus, Legionella pneumophila, Yersiniapestis, Streptococcus (types A and B), Pneumococcus, Meningococcus, Hemophilus influenza (type b), Toxoplasma gondic, Complylobacteriosis, Moraxella catarrhalis, Donovaniosis, and Acdiaomycosis; fungal pathogens including Candidiasis and Aspergillosis; parasitic pathogens including Taenia, Flukes, Roundwormns, Amebiasis, Giardiasis, Cryptosporidium, Schistosoma, Pneumocystis carinii, Trichomoniasis and Trichinosis. Thus, the present invention can also be used to provide a suitable immune response against numerous veterinary diseases, such as Foot and Mouth diseases, Coronavirus, Pasteurella multocida, Helicobacter, Strongylus vulgaris, Actinobacillus pleuropneumonia, Bovine viral diarrhea virus (BVDV), Klebsiella pneumoniae, E. coli, Bordetella pertussis, Bordetella parapertussis and brochiseptica.

Typically, a nucleotide sequence corresponding to one or more of the above-listed antigen(s) is used in the production of the polynucleotides, as described below.

Isolation of Genes and Construction of Polynucleotides

The present invention provides polynucleotides encoding at least one antigen (e.g., antigens derived derived from and/or expressed by viruses, bacteria, fungi, worms, toxins, allergens or cancer cells) operably linked to a non-viral cell- or tissue-specific promoter (e.g., a promoter derived from a regulatory element which controls transcription of a sequence encoding a co-stimulatory molecule). These polynucleotides are useful in eliciting an immune response to the antigen(s), particularly in activating T-lymphocytes.

Nucleotide sequences selected for use in the present invention can be derived from known sources, for example, by isolating the same from cells containing a desired gene or nucleotide sequence using standard techniques. Similarly, the nucleotide sequences can be generated synthetically using standard modes of polynucleotide synthesis that are well known in the art. See, e.g., Edge et al. (1981) Nature 292:756-762; Nambair et al. (1994) Science 223:1299-1301; Jay et al. (1984) J. Biol. Chem. 259:6311-6317. Generally, synthetic oligonucleotides can be prepared by either the phosphotriester method as described by Edge et al., supra, and Duckworth et al. (1981) Nucleic Acids Res. 9:1691-1706, or the phosphoramidite method as described by Beaucage et al. (1981) Tet. Letts. 22:1859, and Matteucci et al. (1981) J. Am. Chem. Soc. 103:3185. Synthetic oligonucleotides can also be prepared using commercially available automated oligonucleotide synthesizers. The nucleotide sequences can thus be designed with appropriate codons for a particular amino acid sequence. In general, one will select preferred codons for expression in the intended host. The complete sequence is assembled from overlapping oligonucleotides prepared by standard methods and assembled into a complete coding sequence. See, e.g., Edge et al. (supra); Nambair et al. (supra) and Jay et al. (supra).

Another method for obtaining nucleic acid sequences for use herein is by recombinant means. Thus, a desired nucleotide sequence can be excised from a plasmid carrying the same using standard restriction enzymes and procedures. Site specific DNA cleavage is performed by treating with the suitable restriction enzyme (or enzymes) under conditions which are generally understood in the art, and the particulars of which are specified by manufacturers of commercially available restriction enzymes. If desired, size separation of the cleaved fragments may be performed by polyacrylamide gel or agarose gel electrophoresis using standard techniques.

Restriction cleaved fragments may be blunt ended by treating with the large fragment of E. coli DNA polymerase I (Klenow) in the presence of the four deoxynucleotide triphosphates (dNTPs) using standard techniques. The Klenow fragment fills in at 5′ single-stranded overhangs but digests protruding 3′ single strands, even though the four dNTPs are present. If desired, selective repair can be performed by supplying only one, or several, selected dNTPs within the limitations dictated by the nature of the overhang. After Klenow treatment, the mixture can be extracted with e.g. phenol/chloroform, and ethanol precipitated. Treatment under appropriate conditions with S1 nuclease or BAL-31 results in hydrolysis of any single-stranded portion.

Yet another convenient method for isolating specific nucleic acid molecules is by the polymerase chain reaction (PCR). Mullis et al. (1987) Methods Enzymol. 155:335-350. This technique uses DNA polymerase, usually a thermostable DNA polymerase, to replicate a desired region of DNA. The region of DNA to be replicated is identified by oligonucleotides of specified sequence complementary to opposite ends and opposite strands of the desired DNA to prime the replication reaction. The product of the first round of replication is itself a template for subsequent replication, thus repeated successive cycles of replication result in geometric amplification of the DNA fragment delimited by the primer pair used. This method also allows for the facile addition of nucleotide sequences onto the ends of the DNA product by incorporating these added sequences onto the oligonucleotide primers (see, e.g., PCR Protocols, A Guide to Methods and Applications, Innis et al (eds) Harcourt Brace Jovanovich Publishers, NY (1994)). PCR conditions used for each amplification reaction are empirically determined. A number of parameters influence the success of a reaction. Among them are annealing temperature and time, extension time, Mg²⁺ and ATP concentration, pH, and the relative concentration of primers, templates, and deoxyribonucleotides. One example of suitable PCR conditions is found below in the Examples.

Once coding sequences for desired proteins have been prepared or isolated, such sequences can be cloned into any suitable vector or replicon. Numerous cloning vectors are known to those of skill in the art, and the selection of an appropriate cloning vector is a matter of choice. Ligations to other sequences are performed using standard procedures, known in the art.

As described in detail below, selected nucleotide sequences can be placed under the control of regulatory sequences such as a promoter, so that the sequence encoding the desired protein is transcribed into RNA in the host tissue transformed by a vector containing this expression construct.

Promoters

The choice of promoter is central to the construction of certain polynucleotides described herein. Thus, the invention provides for expression of a selected antigen driven by a non-viral, preferably mammalian, cell- (or tissue-) specific promoter. In a preferred embodiment, the promoter is derived from a regulatory sequence which controls transcription of a co-stimulatory molecule for example, a promoter derived from a CD80 (also known as B7-1), CD86 (also known as B7-2), CD40 or CD54 gene. Other suitable promoters can be readily determined using the teachings herein.

Genomic organization, including promoter mapping, of suitable co-stimulatory factors, such as CD80 and CD86 has been described, for example in Zhang et al. (1996) Gene 183:1-6; Selvakumar et al. (1993) Immunogenetics 38:292-295 and Fong et al. (1996) J. Immunol. 157:4442-4450. These studies have shown that the CD80 (B7-1) gene promoter consists of three positively regulated regions: a distal region from −2597 to −1555 that contains putative transcription factor binding sites; a proximal region from −130 to −110 that contains a tandem, repeat sequence and a downstream region from +269 to +25 (Zhang et al., supra). Transactions from nucleotide position −806 to −84 have been shown to result in increased transcription activity in CD80-expressing Raji cells and a regulatory element around −41 has been identified (Fong et al., supra). Further mapping of the CD80 promoter or other co-stimulatory molecule promoters can be conducted using methods known in the art in view of these references and the teachings of this specification.

The present invention also provides methods of eliciting an immune response using an immunizing agent in combination with at least one cytokine that enhances the stimulatory lifespan of APCs (e.g., dendritic cells). In these embodiments, either the immunizing agent or the cytokine(s) can be delivered to the subject as a polynucleotide encoding the polypeptide of interest. These polynucleotides can include a wide-variety of promoters, including, for example, constitutive promoters (e.g., CMV, SV-40, housekeeping gene promoters, and the like), inducible promoters (e.g., metallothionine, heat-shock, cytochrome, protein tyroisne kinase, nitric oxide synthase promoters, and the like), and tissue or cell-specific promoters (e.g., CD80/86, muscle creatine kinase, and the like).

In addition to promoters, it may be desirable to add other regulatory sequences which allow for regulation of the expression of protein sequences encoded by the delivered nucleotide sequences. Suitable additional regulatory sequences are known to those of skill in the art, and examples include those which cause the expression of a coding sequence to be turned on or off in response to a chemical or physical stimulus, including the presence of a regulatory compound. Other types of regulatory elements may also be present in the vector, for example, enhancer sequences.

An expression vector is constructed so that the particular coding sequence is located in the vector with the appropriate regulatory sequences such that the positioning and orientation of the coding sequence with respect to the control sequences allows the coding sequence to be transcribed under the “control” of the control sequences (i.e., RNA polymerase, which binds to the DNA molecule at the control sequences, transcribes the coding sequence). Modifications of the sequences encoding the particular protein of interest may be desirable to achieve this end. For example, in some cases it may be necessary to modify the sequence so that it is attached to the control sequences with the appropriate orientation; i.e., to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site.

Generally, nucleic acid molecules used in the subject methods contain coding regions with suitable control sequences and, optionally, ancillary nucleotide sequences which encode cytokines or other immune enhancing polypeptides. The nucleic acid molecules are generally prepared in the form of vectors which include the necessary elements to direct transcription and translation in a recipient cell.

Adjuvants

In order to augment an immune response in a subject, the compositions and methods described herein can further include ancillary substances (e.g., adjuvants), such as pharmacological agents, cytokines, or the like. Suitable adjuvants include any substance that enhances the immune response of the subject to the polynucleotides of the invention. Non-limiting examples include cytokines, e.g., Flt3 ligand, CD40L and TRANCE. As detailed above, these cytokines may enhance the immune response by affecting any number of pathways, for example, by stabilizing the antigen/MHC complex, by causing more antigen/MHC complex to be present on the cell surface, by enhancing maturation of APCs, or by prolonging the life of APCs (e.g., inhibiting apoptosis). For instance, recent studios suggest that CD40L and Flt3 ligand can serve as adjuvants in mouse models (Gurunathan et al., (1998) J. Immunol. 161:4563 and Pulendran et al. (1998), J. Immunol. 188:2075). Wong et al. (1997) J. Exp. Med. 186:2075 reports that TRANCE may promote the life-span of mature dendritic cells. As described herein, these cytokines, delivered as either peptides or as polynucleotides encoding functional peptides, are also be useful in eliciting immune responses.

Ancillary substances may be administered, for example, as proteins or other macromolecules at the same time, prior to, or subsequent to, administration of the co-stimulatory molecule promoter driven polynucleotides, peptide antigens or polynucleotides encoding an antigen of interest. Cytokines can be obtained from a variety of sources, for example Immunex (Seattle, Wash.), Genentech (South San Francisco, Calif.) and Amgen (Thousand Oaks, Calif.). Alternatively, cytokines can be produced using a variety of methods known to those skilled in the art in view of the teachings of this specification. In particular, cytokines can be isolated directly from native sources, using standard purification techniques. Alternatively, the cytokines can be recombinantly produced using expression systems as described above and purified using known techniques. The cytokines can also be synthesized, based on known amino acid sequences or amino acid sequences derived from DNA sequence of a molecule of interest, via chemical polymer syntheses such as solid phase peptide synthesis. Such methods are described for example, in J. M. Stewart and J. D. Young, Solid Phase Peptide Synthesis, 2nd ed, Pierce Chemical Co., Rockford Ill. (1984) and G. Barany and R. B. Merrifield, The Peptides: Analysis, Synthesis, Biology, editors E. Gross and J. Meinenhofer, vol. 2, Academic Press, New York, (1980), pp. 3-254, for solid phase peptide synthesis techniques.

Alternatively, ancillary nucleic acid sequences coding for peptides known to stimulate, modify, or modulate a host's immune response (e.g., cytokines), can be co-administered as polynucleotides with the above-described antigen-encoding polynucleotides or peptide antigens. The gene sequences for a number of these cytokines are known. (see, e.g., GenBank and other publically available databases; Wong et al. (1997) J Biol Chem 272(40):25190-4 (TRANCE); Lyman et al. (1995) Oncogene 11(6):1165-72 (flt3); Spriggs et al., (1992) J. Exp. Med. 176:1543-1550 and Armitage et al. (1992) Nature 357:80-82 (CD40L); Spriggs (1992) Immunol Ser. 56:3-34 (TNF-alpha); Morgan et al. (1976) Science 193:1007-1008 (IL-2); U.S. Pat. No. 5,187,077 (LIF); Brankenhoffet al. (1987) Immunol. 139:4116-4121 (IL-6); etc).

Thus, suitable cytokines can be supplied by administering a polynucleotide encoding the cytokine (or encoding an active fragment thereof). These cytokine-encoding nucleotides can be administered either on the same vector that carries the antigen-encoding sequence, or, alternatively on a separate vector. In some cases, it may be desirable to design a polynucleotide in which both the antigen-encoding sequence and the cytokine-encoding sequence are under the control of the same promoter, for instance one derived from a co-stimulatory molecule. In other cases, it may be desirable to use a cytokine-encoding sequence under the control of a different promoter, for example a constitutive promoter.

Administration of Polynucleotides and Adjuvants

The polynucleotides and ancillary substances described herein may be administered by any suitable method. In a preferred embodiment, described below, the polynucleotides are administered by coating them onto particles and then administering the particles to the subject or cells. However, the polynucleotides may also be delivered using a viral vector as known in the art, or by using non-viral systems, as described for example in U.S. Pat. No. 5,589,466.

Viral Vectors

A number of viral based systems have been used for gene delivery. For example, retroviral systems are known and generally employ packaging lines which have an integrated defective provirus (the “helper”) that expresses all of the genes of the virus but cannot package its own genome due to a deletion of the packaging signal, known as the psi sequence. Thus, the cell line produces empty viral shells. Producer lines can be derived from the packaging lines which, in addition to the helper, contain a viral vector which includes sequences required in cis for replication and packaging of the virus, known as the long terminal repeats (LTRs). The gene of interest can be inserted in the vector and packaged in the viral shells synthesized by the retroviral helper. The recombinant virus can then be isolated and delivered to a subject. (See, e.g., U.S. Pat. No. 5,219,740.) Representative retroviral vectors include but are not limited to vectors such as the LHL, N2, LNSAL, LSHL and LHL2 vectors described in e.g., U.S. Pat. No. 5,219,740, incorporated herein by reference in its entirety, as well as derivatives of these vectors, such as the modified N2 vector described herein. Retroviral vectors can be constructed using techniques well known in the art. See, e.g., U.S. Pat. No. 5,219,740; Mann et al. (1983) Cell 33:153-159.

Adenovirus based systems have been developed for gene delivery and are suitable for delivering the polynucleotides described herein. Human adenoviruses are double-stranded DNA viruses which enter cells by receptor-mediated endocytosis. These viruses are particularly well suited for gene transfer because they are easy to grow and manipulate and they exhibit a broad host range in vivo and in vitro. For example, adenoviruses can infect human cells of hematopoietic, lymphoid and myeloid origin. Furthermore, adenoviruses infect quiescent as well as replicating target cells. Unlike retroviruses which integrate into the host genome, adenoviruses persist extrachromosomally thus minimizing the risks associated with insertional mutagenesis. The virus is easily produced at high titers and is stable so that it can be purified and stored. Even in the replication-competent form, adenoviruses cause only low level morbidity and are not associated with human malignancies. Accordingly, adenovirus vectors have been developed which make use of these advantages. For a description of adenovirus vectors and their uses see, e.g., Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; Rich et al. (1993) Human Gene Therapy 4:461-476.

Adeno-associated viral vector (AAV) can also be used to administer the polynucleotides described herein. AAV vectors can be derived from any AAV serotype, including without limitation, AAV-1, AAV-2, AAV-3, AAV-4, AAV-5, AAVX7, etc. AAV vectors can have one or more of the AAV wild-type genes deleted in whole or part, preferably the rep and/or cap genes, but retain one or more functional flanking inverted terminal repeat (ITR) sequences. Functional ITR sequences are necessary for the rescue, replication and packaging of the AAV virion. Thus, an AAV vector includes at least those sequences required in cis for replication and packaging (e.g., functional ITRs) of the virus. The ITR sequence need not be the wild-type nucleotide sequence, and may be altered, e.g., by the insertion, deletion or substitution of nucleotides, so long as the sequence provides for functional rescue, replication and packaging.

AAV expression vectors are constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is bounded (5′ and 3′) with functional AAV ITR sequences. Suitable AAV constructs can be designed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:1 65-169; and Zhou et al. (1994) J. Exp. Med. 179:1867-1875.

Conventional Pharmaceutical Preparations

Formulation of a preparation comprising the polynucleotides of the present invention, with or without addition of an adjuvant composition, can be carried out using standard pharmaceutical formulation chemistries and methodologies all of which are readily available to the ordinarily skilled artisan. For example, compositions containing one or more nucleic acid molecules (e.g., present in a plasmid or viral vector) can be combined with one or more pharmaceutically acceptable excipients or vehicles to provide a liquid preparation.

Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that the preparation will contain a pharmaceutically acceptable excipient that serves as a stabilizer, particularly for peptide, protein or other like molecules if they are to be included in the vaccine composition. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. A thorough discussion of pharmaceutically acceptable excipients, vehicles and auxiliary substances is available in REMINGTONS PHARMACEUTICAL SCIENCES (Mack Pub. Co., N.J. 1991), incorporated herein by reference.

Certain facilitators of nucleic acid uptake and/or expression (“transfection facilitating agents”) can also be included in, e.g., non-viral vector compositions, for example, facilitators such as bupivacaine, cardiotoxin and sucrose, and transfection facilitating vehicles such as liposomal or lipid preparations that are routinely used to deliver nucleic acid molecules. Anionic and neutral liposomes are widely available and well known for delivering nucleic acid molecules (see, e.g., Liposomes: A Practical Approach, (1990) RPC New Ed., IRL Press). Cationic lipid preparations are also well known vehicles for use in delivery of nucleic acid molecules. Suitable lipid preparations include DOTMA (N-[1-(2,3-dioleyloxy)propyl]-N,N,N-trimethylammonium chloride), available under the tradename Lipofectin.™., and DOTAP (1,2-bis(oleyloxy)-3-(trimethylammonio)propane), see, e.g., Felgner et al. (1987) Proc. Natl. Acad. Sci. USA 84:7413-7416; Malone et al. (1989) Proc. Natl. Acad. Sci. USA 86:6077-6081; U.S. Pat. Nos. 5,283,185 and 5,527,928, and International Publication Nos WO 90/11092, WO 91/15501 and WO 95/26356. These cationic lipids may preferably be used in association with a neutral lipid, for example DOPE (dioleyl phosphatidylethanolamine)-. Still further transfection-facilitating compositions that can be added to the above lipid or liposome preparations include spermine derivatives (see, e.g., International Publication No. WO 93/18759) and membrane-permeabilizing compounds such as GALA, Gramicidine S and cationic bile salts (see, e.g., International Publication No. WO 93/19768).

Alternatively, the nucleic acid molecules of the present invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, speimine, spermidine, as well as conjugates of these molecules.

The formulated vaccine compositions will thus typically include a polynucleotide (e.g., a plasmid) containing a sequence encoding an antigen of interest in an amount sufficient to mount an immunological response. An appropriate effective amount can be readily determined by one of skill in the art. Such an amount will fall in a relatively broad range that can be determined through routine trials. For example, immune responses have been obtained using as little as 1 μg of DNA, while in other administrations, up to 2 mg of DNA has been used. It is generally expected that an effective dose of polynucleotides containing the genomic fragments will fall within a range of about 10 μg to 1000 μg, however, doses above and below this range may also be found effective. The compositions may thus contain from about 0.1% to about 99.9% of the polynucleotide molecules.

Administration of Conventional Pharmaceutical Preparations

Administration of the above-described pharmaceutical preparations can be effected in one dose, continuously or intermittently throughout the course of treatment. Delivery will most typically be via conventional needle and syringe for the liquid compositions and for liquid suspensions containing particulate compositions. In addition, various liquid jet injectors are known in the art and may be employed to administer the present compositions. Methods of determining the most effective means and dosages of administration are well known to those of skill in the art and will vary with the delivery vehicle, the composition of the therapy, the target cells, and the subject being treated. Single and multiple administrations can be carried out with the dose level and pattern being selected by the attending physician. It should be understood that more than one antigen sequence can be carried by a polynucleotide vector construct. Alternatively, separate vectors (e.g., plasmid or viral vectors), each containing sequences expressing one or more antigens can also be delivered to a subject as described herein.

Furthermore, it is also intended that the polynucleotides delivered by the methods of the present invention may be combined with other suitable compositions and therapies. For instance, in order to augment an immune response irk a subject, the compositions and methods described herein can further include ancillary substances (e.g., adjuvants), such as phlanacological agents, cytokines, or the like. Ancillary substances may be administered, for example, as proteins or other macromolecules at the same time, prior to, or subsequent to, administration of the polynucleotides described herein. The nucleic acid molecule compositions may also be administered directly to the subject or, alternatively, delivered ex vivo, to cells derived from the subject, using methods known to those skilled in the art.

Coated Particles

In one embodiment, the polynucleotides (e.g., DNA vaccines) and/or adjuvants are delivered using carrier particles (e.g., core carriers). Particle-mediated methods for delivering such nucleic acid preparations are known in the art. Thus, once prepared and suitably purified, the above-described nucleic acid molecules and/or adjuvants can be coated onto carrier particles (e.g., core carriers) using a variety of techniques known in the art. Carrier particles are selected from materials which have a suitable density in the range of particle sizes typically used for intracellular delivery from an appropriate particle-mediated delivery device. The optimum carrier particle size will, of course, depend on the diameter of the target cells. Alternatively, colloidal gold particles can be used wherein the coated colloidal gold is administered (e.g., injected) into tissue (e.g., skin or muscle) and subsequently taken-up by immune-competent cells.

For the purposes of the invention, tungsten, gold, platinum and iridium caiier particles can be used. Tungsten and gold particles are preferred. Tungsten particles are readily available in average sizes of 0.5 to 2.0 μm in diameter. Although such particles have optimal density for use in particle acceleration delivery methods, and allow highly efficient coating with DNA, tungsten may potentially be toxic to certain cell types. Gold particles or microcrystalline gold (e.g., gold powder A1570, available from Engelhard Corp., East Newark, N.J.) will also find use with the present methods. Gold particles provide uniformity in size (available from Alpha Chemicals in particle sizes of 1-3 μm, or available from Degussa, South Plainfield, N.J. in a range of particle sizes including 0.95 μm) and reduced toxicity.

A number of methods are known and have been described for coating or precipitating DNA or RNA onto gold or tungsten particles. Most such methods generally combine a predetermined amount of gold or tungsten with plasmid DNA, CaCl₂ and spermidine. The resulting solution is vortexed continually during the coating procedure to ensure uniformity of the reaction mixture. After precipitation of the nucleic acid, the coated particles can be transferred to suitable membranes and allowed to dry prior to use, coated onto surfaces of a sample module or cassette, or loaded into a delivery cassette for use in a suitable particle delivery instrument.

Peptide adjuvants (e.g., cytokines), can also be coated onto suitable carrier particles, e.g., gold or tungsten. For example, peptides can be attached to the carrier particle by simply mixing the two components in an empirically determined ratio, by ammonium sulfate precipitation or other solvent precipitation methods familiar to those skilled in the art, or by chemical coupling of the peptide to the carrier particle. The coupling of L-cysteine residues to gold has been previously described (Brown et al., Chemical Society Reviews 9:271-311 (1980)). Other methods include, for example, dissolving the peptide antigen in absolute ethanol, water, or an alcohol/water mixture, adding the solution to a quantity of carrier particles, and then drying the mixture under a stream of air or nitrogen gas while vortexing. Alternatively, the peptide antigens can be dried onto carrier particles by centrifugation under vacuum. Once dried, the coated particles can be resuspended in a suitable solvent (e.g., ethyl acetate or acetone), and triturated (e.g., by sonication) to provide a substantially uniform suspension.

Administration of Coated Particles

Following their formation, carrier particles coated with either nucleic acid preparations, or peptide or protein adjuvant preparations, are delivered to a subject, for example transdermally, using particle-mediated delivery techniques.

Various particle delivery devices suitable for particle-mediated delivery techniques are known in the art, and are all suited for use in the practice of the invention. Current device designs employ an explosive, electric or gaseous discharge to propel the coated core carrier particles toward target cells. The coated particles can themselves be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of an electric discharge-type particle acceleration apparatus is described in U.S. Pat. No. 5,120,657. Another electric discharge apparatus suitable for use herein is described in U.S. Pat. No. 5,149,655. The disclosure of all of these patents is incorporated herein by reference in their entireties.

The coated particles are administered to the subject to be treated in a manner compatible with the dosage formulation, and in an amount that will be effective to bring about a desired immune response. The amount of the composition to be delivered which, in the case of nucleic acid molecules is generally in the range of from 0.001 to 100.0 μg, more typically 0.01 to 10.0 μg of nucleic acid molecule per dose, and in the case of peptide or protein molecules is 1 μg to 5 mg, more typically 1 to 50 μg of peptide, depends on the subject to be treated. The exact amount necessary will vary depending on the age and general condition of the individual being immunized and the particular nucleotide sequence or peptide selected, as well as other factors. An appropriate effective amount can be readily determined by one of skill in the art upon reading the instant specification.

Thus, an effective amount of the antigens herein described, or nucleic acids coding therefor, will be sufficient to bring about a suitable immune response in an immunized subject, and will fall in a relatively broad range that can be determined through routine trials. Preferably, the coated particles are delivered to suitable recipient cells in order to bring about an immune response (e.g., T-cell activation) in the treated subject.

Particulate Compositions

Alternatively, the antigen of interest (as well as one or more selected adjuvant) can be formulated as a particulate composition. More particularly, formulation of particles comprising the antigen and/or adjuvant of interest can be carried out using standard pharmaceutical formulation chemistries and methodologies all of which are readily available to the reasonably skilled artisan. For example, one or more antigen and/or adjuvant can be combined with one or more pharmaceutically acceptable excipient or vehicle to provide an antigen, adjuvant, or vaccine composition. Auxiliary substances, such as wetting or emulsifying agents, pH buffering substances, and the like, may be present in the excipient or vehicle. These excipients, vehicles and auxiliary substances are generally pharmaceutical agents that do not themselves induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that an antigen composition will contain a pharmaceutically acceptable carrier that serves as a stabilizer, particularly for peptide, protein or other like antigens. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. A thorough discussion of pharmaceutically acceptable excipients, carriers, stabilizers and other auxiliary substances is available in REMINGTONS PHARMACEUTICAL SCIENCES (Mack Pub. Co., N.J. 1991), incorporated herein by reference.

The formulated compositions will include an amount of the antigen of interest which is sufficient to mount an immunological response, as defined above. An appropriate effective amount can be readily determined by one of skill in the art. Such an amount will fall in a relatively broad range, generally within the range of about 0.1 μg to 25 mg or more of the antigen of interest, and specific suitable amounts can be determined through routine trials. The compositions may contain from about 0.1% to about 99.9% of the antigen. If an adjuvant is included in the composition, or the methods are used to provide a particulate adjuvant composition, the adjuvant will be present in a suitable amount as described above. The compositions are then prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be densified using the techniques described in commonly owned International Publication No. WO 97/48485, incorporated herein by reference.

These methods can be used to obtain nucleic acid particles having a size ranging from about 0.1 to about 250 μm, preferably about 10 to about 150 μm, and most preferably about 20 to about 60 μm; and a particle density ranging from about 0.1 to about 25 g/cm³, and a bulk density of about 0.5 to about 3.0 g/cm³, or greater.

Similarly, particles of selected adjuvants having a size ranging from about 0.1 to about 250 μm, preferably about 0.1 to about 150 μm, and most preferably about 20 to about 60 μm; a particle density ranging from about 0.1 to about 25 g/cm³, and a bulk density of preferably about 0.5 to about 3.0 g/cm³, and most preferably about 0.8 to about 1.5 g/cm³ can be obtained.

Single unit dosages or multidose containers, in which the particles may be packaged prior to use, can comprise a hermetically sealed container enclosing a suitable amount of the particles comprising the antigen of interest and/or the selected adjuvant (e.g., the vaccine composition). The particulate compositions can be packaged as a sterile formulation, and the hermetically sealed container can thus be designed to preserve sterility of the formulation until use in the methods of the invention. If desired, the containers can be adapted for direct use in a needleless syringe system, and can take the form of capsules, foil pouches, sachets, cassettes, and the like.

The container in which the particles are packaged can further be labeled to identify the composition and provide relevant dosage information. In addition, the container can be labeled with a notice in the form prescribed by a governmental agency, for example the Food and Drug Administration, wherein the notice indicates approval by the agency under Federal law of the manufacture, use or sale of the antigen, adjuvant (or vaccine composition) contained therein for human administration.

Administration of Particulate Compositions

Following their formation, the particulate composition (e.g., powder) can be delivered transdermally to vertebrate tissue using a suitable transdermal particle delivery technique. Various particle delivery devices suitable for administering the substance of interest are known in the art, and will find use in the practice of the invention. A particularly preferred transdermal particle delivery system employs a needleless syringe to fire solid particles in controlled doses into and through intact skin and tissue. See, e.g., U.S. Pat. No. 5,630,796 to Bellhouse et al. which describes a needleless syringe (also known as “the PowderJect® particle delivery device”). Other needleless syringe configurations are known in the art and are described herein.

The particulate compositions (comprising the antigen of interest and optionally a selected adjuvant) can then be administered using a transdermal delivery technique. Preferably, the particulate compositions will be delivered via a powder injection method, e.g., delivered from a needleless syringe such as those described in commonly owned International Publication Nos. WO 94/24263, WO 96/04947, WO 96/12513, and WO 96/20022, all of which are incorporated herein by reference. Delivery of particles from such particle delivery devices is practiced with particles having an approximate size generally ranging from 0.1 to 250 μm, preferably ranging from about 10-70 μm. Particles larger than about 250 μm can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, and injection velocities generally range between about 100 and 3,000 m/sec, or greater. With appropriate gas pressure, particles having an average diameter of 10-70 μm can be accelerated through the nozzle at velocities approaching the supersonic speeds of a driving gas flow.

If desired, these particle delivery devices (e.g., a needleless syringe) can be provided in a preloaded condition containing a suitable dosage of the particles comprising the antigen of interest and/or the selected adjuvant. The loaded syringe can be packaged in a hermetically sealed container, which may further be labeled as described above.

Compositions containing a therapeutically effective amount of the powdered molecules described herein can be delivered to any suitable target tissue via the above-described particle delivery devices. For example, the compositions can be delivered to muscle, skin, brain, lung, liver, spleen, bone marrow, thymus, heart, lymph, blood, bone cartilage, pancreas, kidney, gall bladder, stomach, intestine, testis, ovary, uterus, rectum, nervous system, eye, gland and connective tissues. For nucleic acid molecules, delivery is preferably to and the molecules expressed in, terminally differentiated cells; however, the molecules can also be delivered to non-differentiated, or partially differentiated cells such as stem cells of blood and skin fibroblasts.

The powdered compositions are administered to the subject to be treated in a manner compatible with the dosage formulation, and in an amount that will be prophylactically and/or therapeutically effective. The amount of the composition to be delivered, generally in the range of from 0.5 μg/kg to 100 μg/kg of nucleic acid molecule per dose, depends on the subject to be treated. Doses may be as low as 0.5 μg for 50 kg subject, or approximately 0.01 μg/kg. Doses for other pharmaceuticals, such as physiological active peptides and proteins, generally range from about 0.1 μg to about 20 mg, preferably 10 μg to about 3 mg. The exact amount necessary will vary depending on the age and general condition of the individual to be treated, the severity of the condition being treated, the particular preparation delivered, the site of administration, as well as other factors. An appropriate effective amount can be readily determined by one of skill in the art.

Thus, a “therapeutically effective amount” of the present particulate compositions will be sufficient to bring about treatment or prevention of disease or condition symptoms, and will fall in a relatively broad range that can be determined through routine trials.

Vaccination Regimes

As is apparent to those skilled in the art in view of the teachings of this specification, vaccination with the above-described polynucleotides (DNA vaccines) can be effected in one dose, continuously or intermittently throughout the course of treatment. Methods of determining the most effective means and dosages of administration are well known to those of skill in the art and will vary with the delivery vector, the nature of the composition, the specific therapy sought, the target cells, and the subject being treated. Single and multiple administrations can be carried out with the dose level and pattern being selected by suitable medical personnel. It should be understood that more than one antigen can be expressed by the delivered polynucleotide. Alternatively, separate vectors, each expressing one or more different antigens under the control of a co-stimulatory molecule promoter, can also be delivered to a subject as described herein.

Furthermore, it is also intended that the immunizing antigen (e.g., polynucleotide or peptide) delivered by the methods of the present invention be combined with other suitable compositions and therapies. For instance, a T-cell response may be enhanced by delivering a polynucleotide or peptide described herein with one or more additional agents, such as cytokines, can be administered prior or subsequent to or simultaneously with (1) the polynucleotides having co-stimulatory molecule promoters driving antigen-expression; (2) polynucleotides encoding at least one antigen or (3) peptide antigens. These additional agents can be provided in various forms, for example, as purified molecules, or by vectors encoding full-length or functional fragments of the polypeptide. The vectors may be distinct from those carrying the sequences encoding the antigen(s), or may be carried on the same vector. Whether the cytokine-encoding sequence is on the same or different vector, it's expression can be driven by the same promoter that drives antigen expression or by a different promoter. In this way, APC maturation cytokines such as CD40 ligand (CD40L or CD 154), tumor-necrosis factor-related activation-induced cytokine (TRANCE), and Flt3 ligand (flt-3L), can be provided to enhance the immune response, apparently by increasing expression of co-stimulatory ligands on the APC, stabilizing the antigen/MHC complex or inhibiting apoptosis of the APC.

Below are examples of specific embodiments for carrying out the present invention. The examples are offered for illustrative purposes only, and are not intended to limit the scope of the present invention in any way.

### EXAMPLES

Efforts have been made to ensure accuracy with respect to numbers used (e.g., amounts, temperatures, etc.), but some experimental error and deviation should, of course, be allowed for.

### Example 1

### Nucleic Acid Immunization Using CD80 Promoter Driven Plasmids

In order to assess the specificity and effectiveness of nucleic acid immunization using DNA vaccine plasmids containing CD80 or CD86 promoters, the following studies were carried out.

A. Plasmid Preparation

The DNA sequence of the mouse and human CD80 gene promoter was obtained from the GenBank Database. The DNA primers for synthesizing the mouse CD80 promoter by PCR were obtained from Life Technologies, Gibco BRL, and had the following sequences:

(SEQ ID NO:1)

(1) 5′ -ACG CGT CGA CTC TAG AAG GAG ACA TTC AGC
TG -3′

(SEQ ID NO:2)

(2) 5′- ACG CGT CGA CAG CTT TCA TGG CCT AGC TGC
TA -3′

(SEQ ID NO:3)

(3) 5′ -ATT CGG CCG CGG TCT AGA GCC AAT GGA GCT
TAG G -3′

(SEQ ID NO:4)

(4) 5′- ATT CGG CCG CGG AGA GTT CTG AAT CAG GGT
GT -3′

Similarly, DNA primers for synthesizing human CD80 promoter by PCR were obtained from Life Technologies, Gibco BRL, and had the following sequences:

(SEQ ID NO:5)

(5) 5′- ACG CGT CGA CAG TCT TCC TCA TCC CAC CA -3′

(SEQ ID NO:6)

(6) 5′- ACG CGT CGA CCA TCA CAC AGC AAG GCT AG -3′

(SEQ ID NO:7)

(7) 5′- ACG CGT CGA CGT TTG TTA GTC CAT GCA CG -3′

(SEQ ID NO:8)

(8) 5′- TCC CCG CGG AGA GAG GCG ACA TTT C -3′

Fragments of the CD80 promoter (human and mouse) were amplified by PCR by reacting 200 μg of human or mouse genomic DNA, 1× Turbo™ Pfu buffer (Stratagene, La Jolla, Calif., 20 mM Tris-Cl, pH 8.8, 2 mM MgSO₄, 10 mM KCl, 10 mM (NH₄)₂ SO₄, 0.1% Triton, 0.1 mg/ml nuclease-free BSA), 20 pm 5′ primer, 20 pm 3′ primer, 200 μm dNTPs, 1U Perfect Match.™. PCR enhancer (StrataGene, La Jolla, Calif.) and 2.5 U Pfu Turbo™ (Stratagene) for 35 cycles of: 96° C. for 45 seconds; 55° C. for 45 seconds and 72° C. for 45 seconds. Following completion of the 35 cycles, the PCR products were held at 4° C. The PCR product was purified by QIAquick.™. PCR purification kit (Qiagen Corporation), according to the manufacturer's instructions.

The purified PCR product was double digested with Sal I (New England BioLabs) and Sac II (New England BioLabs) in buffer #3 (100 mM NaCl, 50 mM Tris-Cl, 10 mM MgCl₂, 1 mM DTT, pH 7.9) at 37° C. overnight. The digested PCR product was run on a 1% agarose gel and the correct band was excised from the gel. The DNA in the gel slice was purified using a GenElute™ ethidium bromide spin column (Supelco).

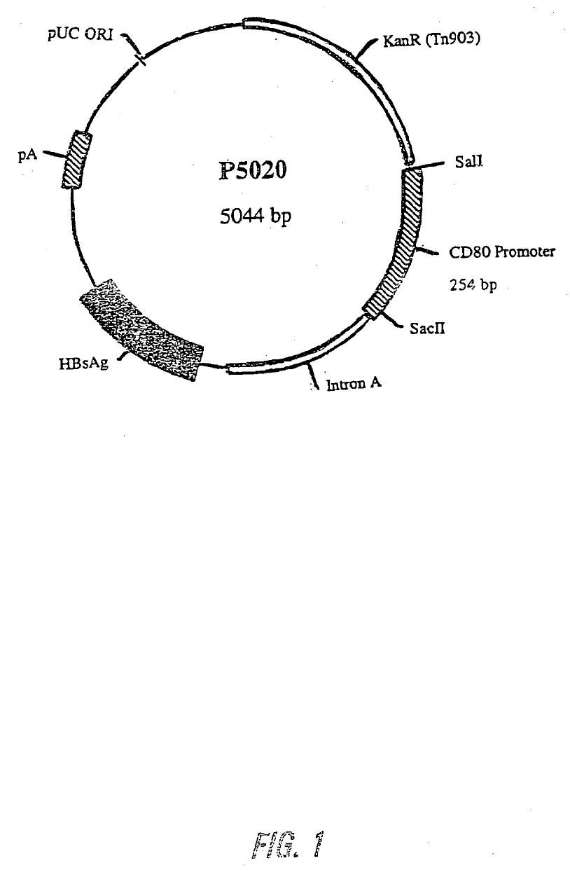

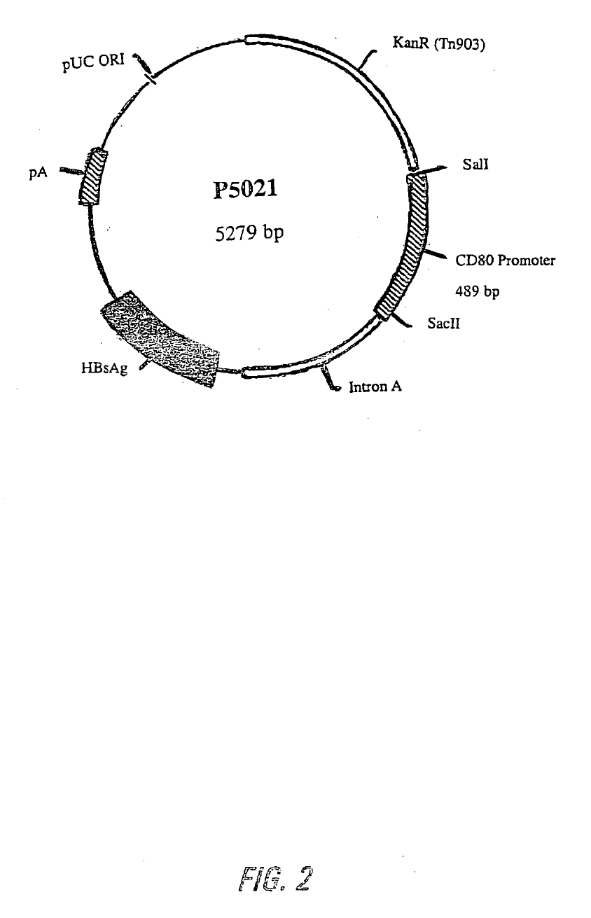

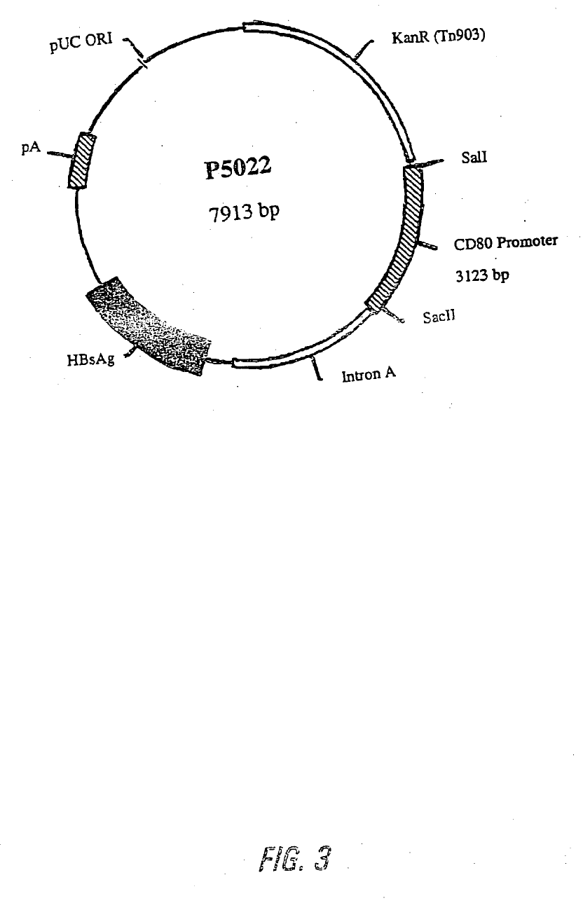

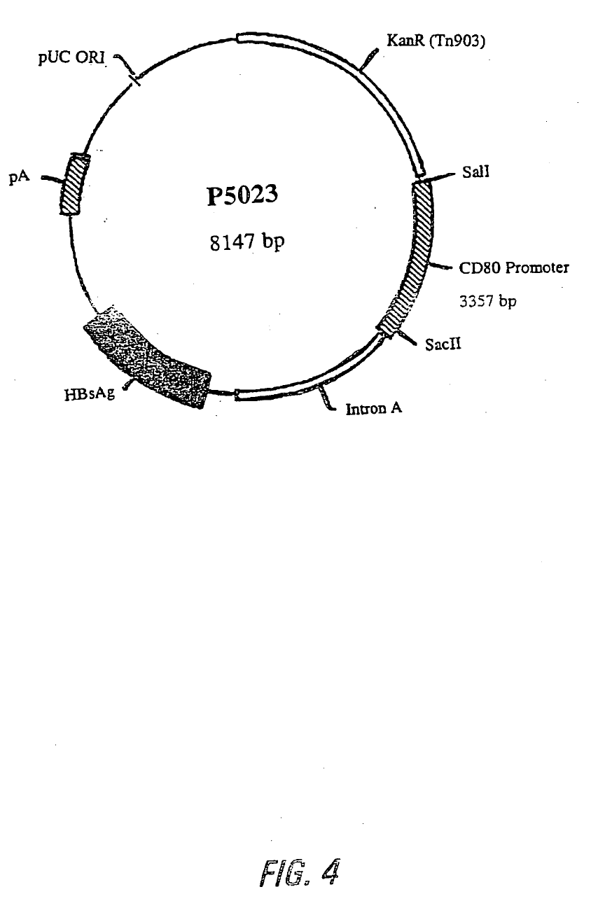

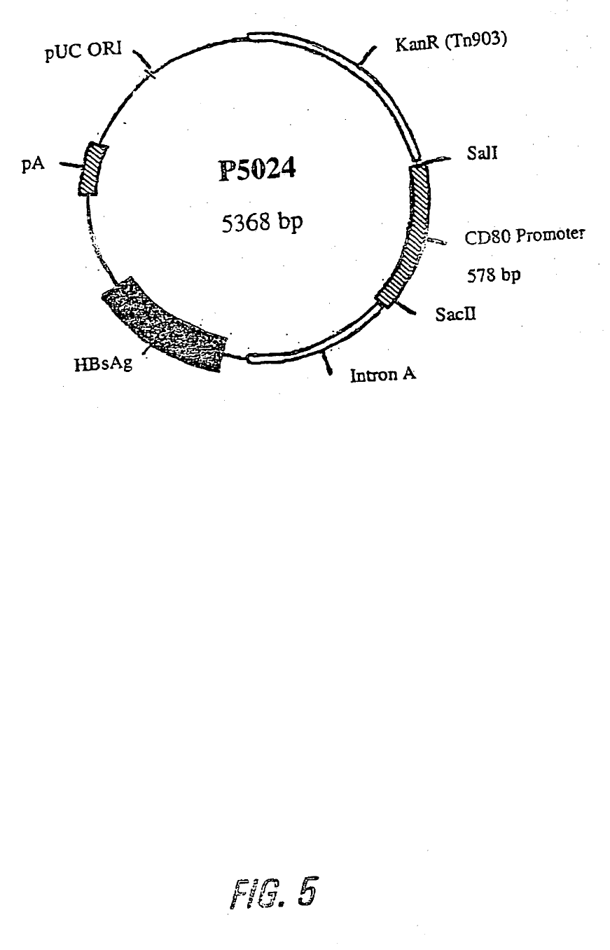

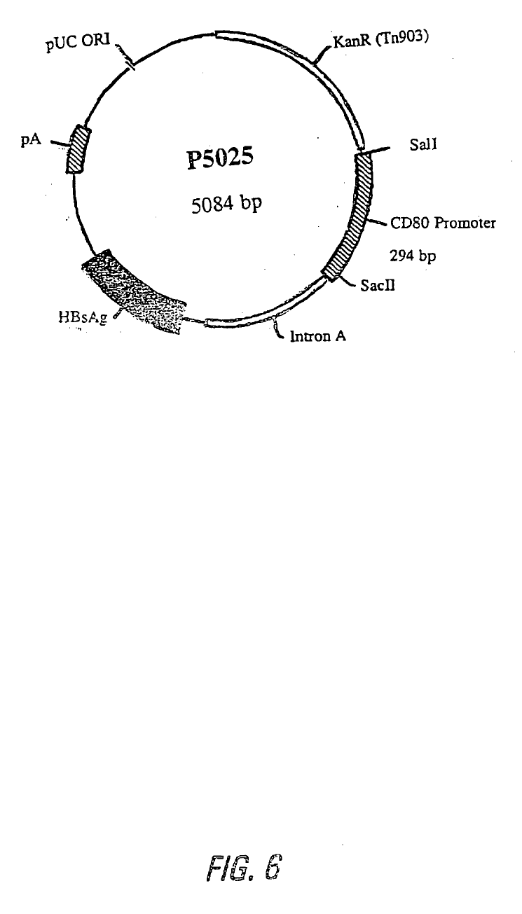

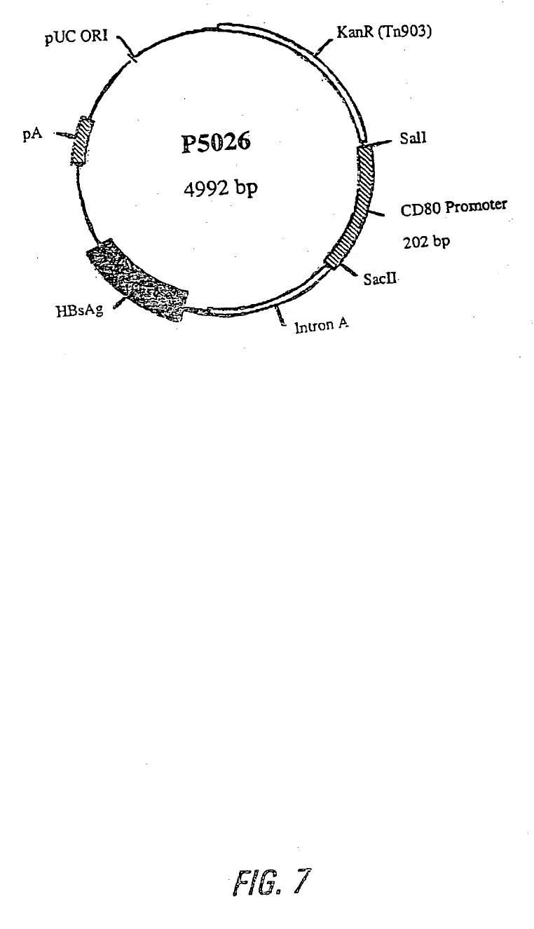

Plasmids were constructed by removing the CMV promoter from plasmid pWRG7128 (Tacket et al. (1999) Vaccine 17:2826) and replacing it with the amplified CD80 promoter segments. The plasmid pWRG7128 contains, in addition to suitable control elements, a sequence encoding the hepatitis B surface antigen (HBsAg) which is under the transcriptional control of a cytomegalovirus (CMV) promoter, and has been shown to produce HbsAg particles upon transfection into most cell types. The pWRG7128 plasmid was constructed as follows. A cloning vector pWkG7077 (Schmaljohn et al. (1997) J. Virol. 71:9563-9569) was prepared to accept a HBsAg coding sequence by digesting the vector to completion with BamH1, followed by a partial digest with Hind3. After blunting the 5′ overhangs by treatment with Klenow fragment and deoxyribonucleotides, the 4.3 kB vector fragment was isolated. The 1.35 kB HbsAg insert fragment (containing the untranslated pre-S2 sequence, the 226 amino acid HbsAg coding sequence of the adw strain, and the HBV enhancer element) was excised from plasmid pAM6 (ATCC, Rockford, Md.) by digesting with BamH1. After blunt-ending by treatment with the Klenow fragment and deoxyribonucleotides, the fragment was isolated and ligated into the 4.3 kB vector fragment described above. The resulting recombinants were screened for proper orientation of the insert and a correct isolate was identified and designated as an intermediate plasmid (pWRG7074). In order to remove the start of the codon of the X protein (present at the 3′ end of the pAM6 1.35 kB insert), a 4.86 kB vector fragment was isolated from the pWRG7074 plasmid by digesting with Bgl2, blunt-ending with the Klenow fragment and deoxyribonucleotides, and then digesting with BstX1. Next, a 754 bp insert fragment was isolated from the pWRG7074 construct by digestion with Nco1, treating with mung bean nuclease, and digesting with BstX1. The resulting vector and insert fragments were then ligated together to form the clinical plasmid pWRG7128. The plasmids constructed are shown in Table 1 and in FIGS. 1-7.

TABLE 1
Representative Plasmids

| | | Length of CD80 promoter |
|---|---|---|
| Plasmid Name (size) | Primers Used | obtained (source) |
| p5020 (5044 bp) | (2) and (4) | 254 bp (mouse) |
| p5021 (5279 bp) | (2) and (3) | 489 bp (mouse) |
| p5022 (7913 bp) | (1) and (4) | 3123 bp (mouse) |
| p5023 (8147 bp) | (1) and (3) | 3357 bp (mouse) |
| p5024 (5368 bp) | (5) and (8) | 578 bp (human) |
| p5025 (5084 bp) | (6) and (8) | 294 bp (human) |
| p5026 (4992 bp) | (7) and (8) | 202 bp (human) |

pWGR7128 was digested with Sal I and Sac II in buffer #3 at 37° C. overnight. The digested vector was run on a 1% agarose gel and the correct vector band was excised from the gel and purified using a GenElute™ ethidium bromide spin column.

To ligate the promoter into the prepared vector, 20 ng of vector and 100 ng of insert promoter were ligand in 1×T4 DNA ligase buffer (50 mM Tris-Cl, 10 mM MgCl₂, 10 mM DTT, 1 mM ATP, 25 μg/ml BSA, pH 7.8) by 6 weiss units T4 DNA ligand (New England Biolabs) at 15° C. overnight.

For selection of properly ligated constructs, the ligation mixture was diluted 1:3 in sterile water. Five μl of the diluted mixture added to 50 μl MAX Efficiency DH5α Competent Cells™ (Gibco-BRL) and incubated on ice for 30 minutes. The transformation mix was heat shocked at 42° C. for 45 seconds and immediately put back on ice for a 2 minute incubation. One ml of SOC media was added to the transformed cells and incubated with shaking at 37° C. for 1 hour. After incubation, the cells were centrifuged briefly, and plated onto a kanamycin-LB plate. The plates were incubated overnight at 37° C.

To confirm successful ligation, single colonies were picked and inoculated in 3.0 ml of LB/Kan media and cultured, with shaking, at 37° C. overnight. Plasmids were isolated from the culture, digested with Sal I and Sac II and visualized on a 1% agarose gel with ethidium bromide staining.

B. Antigen Expression

Two mls B16 cells were seeded in a 6 well plate at 2×10⁵ cells/ml. The cells were cultured in a 37° C. incubation with 5% CO₂ overnight to reach 60-80% confluency. The cells were transfected as follows. Two μg of endotoxin free plasmid Dna in 100 μl Opti-MEM®1 reduced serum media (Gibco-BRL) and 10 ul of lipofectin in 90 ul in Opti-MEM®1 reduced serum media were separately incubated at room temperature for 45 minutes. The two solutions were then mixed and incubated for 15 minutes at room temperature. During this incubation, the B16 cells were washed in serum-free medium 3 times. For each transfection, 0.8 ml of Opti-MEM®1 reduced serum media was added to the mixed solutions and the complex overlaid onto the B16 cells. The cells were then incubated at 37° C. for 5 hours. Subsequently, 1.0 ml 20% FBS medium was added. The medium was collected 48 hours later for antigen expression analysis.

Expression of HBsAg in transfected B16 cells is shown in Table 2. The cells were treated in one of the three following manners: (1) transfection with a positive control, i.e., pWRG7128 which expressed antigen (i.e., HBsAg) in B16 cells; (2) transfection with plasmids p5020 and p5021 as described above and (3) not transfected (negative control).

TABLE 2
In vitro Expression of HBsAg by Cells Transfected
with Various Plasmids

| Cell Culture Groups | Mean Level of Antigen Expression* |
|---|---|
| Non-Transfected Cells (Negative | .0075 |
| Control) | |
| Cells transfected with pWRG7128 | >2.0 |
| Cells transfected with p5020 | 0.0045 |
| Cells transfected with p5021 | 0.0065 |

*Data are presented as the mean OD values (492.6 wavelength) for supernatants obtained from two separate cultures and analyzed using HBV surface antigen kit, Abbott Laboratories Diagnostic Division (Auszyme Monoclonal, List No. 1980-24).

As shown in Table 2, unlike the positive control plasmid pWRG7128, expression of the HBV surface antigen was not detected in cultured B16 cells transfected with plasmids p5020 and p5021. Thus, the CD80 promoter does not drive expression of antigen in non-antigen-presenting cells.

C. Preparation of Coated Microparticles

Plasmid DNA was coated onto 1-3 μm gold particles (Degussa Corp., South Plainfield, N.J.) using techniques described by Eisenbraun et al. (1993) DNA Cell Biol. 12:791-797. Briefly, gold particles were suspended in 50 mM spermnidine and mixed with an equal volume of plasmid in water. This solution was mixed on a vortex and volume of 1 M CaCl₂ half that of the gold/DNA mixture was added dropwise. The mixture was incubated for 10 minutes at room temperature and centrifuged to pellet the particles. The particles were washed 3 times with 100% ethanol and resuspended in ethanol containing 0.05-0.5% polyvinyl pyrrilodine (PVP). The DNA-coated gold particles were then loaded into Tefzel® tubing as described in U.S. Pat. No. 5,584,807 to McCabe, and the tubing was cut into 1.27 cm lengths to serve as cartridges in a PowderJect® XR-1 particle delivery device (PowderJect Vaccines, Inc. Madison, Wis.). The helium-pulse XR-1 particle delivery device has been previously described (see, e.g., U.S. Pat. Nos. 5,584,807 and 5,865,796). In the vaccinations, each 1.27 cm cartridge contained 0.5 mg gold particles coated with 2 μg of plasmid DNA.

D. Antibody Response

Based on the positive results seen in the above-described in vitro transfection study, a vaccination trial was initiated using in vivo particle-mediated delivery methods. Animal subjects receiving nucleic acid immunizations in the present study included: (1) a first experimental group of 4 mice that were vaccinated by particle-mediated delivery to the epidermis with pWGR7128 positive control; and (2) a second experimental group of 6 mice that were vaccinated by particle-mediated delivery to the epidermis with the CD80-promoter driven HBsAg plasmids p5020 or p5021. Blood samples were taken from each animal prior to immunization (nave mice).

Mice were immunized (primed) with plasmids p5020 and p5021 or with the control pWGR7128 by particle-mediated delivery of the plasmid coated onto gold-particles (0.5 mg of gold/shot coated with 2 μg DNA/mg gold). For immunization, mice were shaved and the particles delivered to the abdomen skin using an XR-1 device with research barrel operated at 500 psi of helium. Two shots were given to each mouse per immunization. Four weeks later the animals were boosted, following the same immunization protocol used for the prime immunization.

Serum was collected from the animals prior to immunization, at 2 and 4 weeks after prime, and at 2 weeks after boost. Serum antibody levels were analyzed using the AUSAB EIA kit, Abbott Laboratories Diagnostics Division, according to the directions supplied by the manufacturer. No antibody reactivity against hepatitis B virus (HBV) surface antigen was detected in the sera samples taken from animals immunized with plasmids p5020 and p5021; however, at 2 weeks post boost the serum antibody titers were >3000 mlU/ml in mice immunized with pWGR7128 (Table 3).

TABLE 3
HBsAg Specific Serum Antibody Titers in Mice Immunized
with CD80 Plasmids

| Treatment Group | Mean Level of Antibody Titer* |
|---|---|
| Naive Mice (Negative Control) | 223.3 |
| Mice Immunized with pWRG7128 | >3000.0 |
| Mice Immunized with p5020 | 17.9 |
| Mice Immunized with p5021 | 32.1 |

*Data are presented as the mean HBV surface antigen antibody titer (mlU/ml) for designated groups of mice. Serum antibody titers were determined using the assay kit from Abbott Laboratories Diagnostic Division (AUSAB EIA, List 9006-24).

Thus, CD80 promoter driven plasmids do not cause a rise in mean antibody titers in serum.

E. Cell Mediated Immune Response

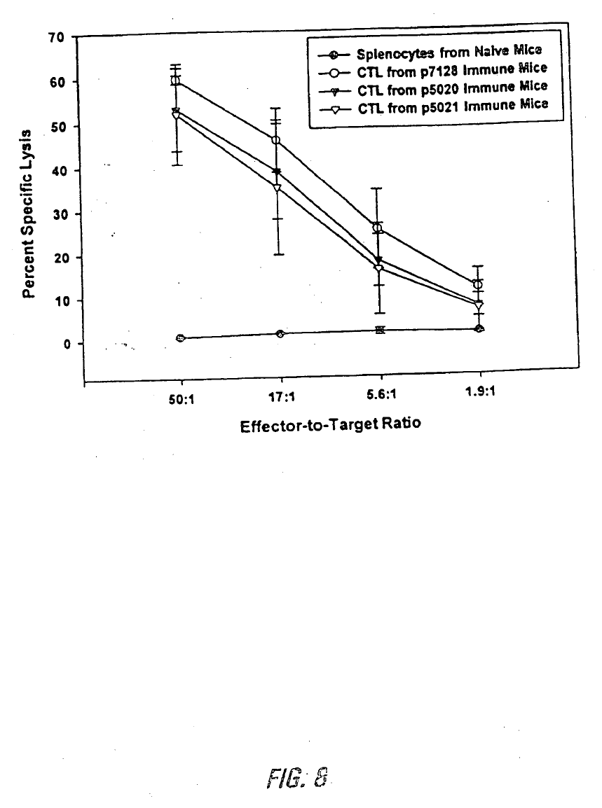

Based on the positive results seen in the above-described analysis of serum antibody titers, a study of cytotoxic T-cell activity in the immunized mice was conducted. Cytotoxic T-cell (CTL) activity was analyzed with splenocytes obtained from immunized mice that were sacrificed at 2 weeks post boost. As shown in FIG. 8, CTL responses elicited by immunization with plasmids p5020 and p5021 were similar to those elicited by plasmid pWRG7128. These responses were much greater than that seen in splenocytes collected from nave mice.

As a result of the above-described studies, it can be seen that nucleic acid immunization provides a T-cell specific immune response where antigen expression is driven by a promoter derived from a co-stimulatory molecule. Moreover, the T-cell response is comparable to that seen using the positive control.

### Example 2

### SIV-Immune Response

In order to determine if an expression vector encoding HBsAg driven by a human CD80 promoter (hCD80-HBsAg) expresses in monkey dendritic cells, the following studies are conducted. Monkey dendritic cells (DCs) are isolated from PBMC, essentially as described in van der Meide et al. (1995) J. Med. Primatol. 24:271-281. The isolated DCs are than transfected, essentially as described in Example 1 for B16 cells with plasmids encoding HBsAg driven by a human CD80 promoter. A non-APC line, for example monkey COS cells are similarly transfected. Expression of HBsAg in the supernatant and/or in cells is conducted by immunohistochemical staining.

In view of the cell-specific expression, studies are conducted to determine the extent of HBsAg expression in APCs. Plasmid hCD80-HBsAg is delivered into the epidermis of monkey skin using a PowderJect® XR particle delivery device. Various additional epidermal sites are also studied. Gold is used as a negative control while CMV-HBsAg is used as a positive control. GM-CSF is administered to test for dendritic cell recruitment. A biopsy is performed on the sites of administration after 24 or 48 hours. The tissue is sectioned and evaluated for HBsAg expression in dendritic cells by immunohistochemical staining using specific antibodies for HBsAg and dendritic cell surface markers. Expression of HBsAg in dendritic and Langerhans cells is evaluated.

A hCD80-SIV expression vector is constructed, for example, by replacing the sequence encoding HBsAg with a suitable SIV-encoding sequence. Monkeys are immunized with the hCD80-SIV construct and compared to monkeys immunized with a CMV-SIV plasmid (positive control). Delivery of the plasmids is performed using the particle delivery device. Antigen expression, antibody response and CTL activation is evaluated, essentially as described above in Example 1. The monkeys are then challenged with a pathogenic SIV and monitored for clinical manifestations of SIV.

### Example 3

### Use of a Cytokine Adjuvant

In order to assess the ability of a polynucleotide encoding a TNF related activation induced kinase (TRANCE) to enhance an immune response against a coadministered antigen sequence, the following studies were carried out.

A. Plasmid Preparation

A cDNA coding sequence for murine TRANCE was derived from the mRNA sequence (GenBank No. AF013170) and cloned into the insertion site of a pFLAG-CMV2 expression vector (Sigma, catalog number E4026) to provide an expression construct containing the TRANCE coding sequence under transcriptional control of the CMV2 promoter. The plasmid construct was termed pTRANCE.

A plasmid containing sequences encoding the hepatitis B core antigen (HBcAg) and hepatitis B surface antigen (HBsAg) was constructed as follows. HBcAg and HBsAg coding sequences were both obtained from the HBV clone pAM6 (ATCC Accession No. 45020). To generate the HBsAg coding region, the pAM6 construct was cut with NcoI and treated with mung bean nuclease to remove the start codon of the X-antigen. The resultant DNA was then cut with BamHI and treated with T4 DNA polymerase to blunt-end the DNA and create an HBsAg expression cassette. The HBsAg expression cassette is present in the 1.2 kB fragment. The plasmid construct pPJV7077 (Schmaljohn et al. (1997) J. Virol. 71:9563-9569) which contains the full-length human CMV (Towne strain) immediate early promoter (with enhancer) was cut with HindIII and BglII, and then treated with T4 DNA polymerase and calf-alkaline phosphatase to create blunt-ended DNA, and the HEsAg expression cassette was ligated into the plasmid to yield the pWRG7128 construct.

To generate the HBcAg coding region, the pAM6 construct was cut to create an HBcAg expression cassette, after which the HBcAg sequence was truncated by site directed mutagenesis to remove the C-terminal arginine-rich region from the core antigen particle (which deletion does not interfere with particle formation). The truncated HBcAg sequence was then cloned into a plasmid construct containing the human elongation factor promoter (“hELF”, Mizushima et al. (1990) Nucl. Acids Res. 18:5322) to provide a HBcAg vector construct.

Expression cassettes containing: (a) the CMV promoter/enhancer, the Intron A-5′ untranslated region, and the human tissue plasminogen activator (hTPA) signal peptide (“CMV-IA-TPA”); or (b) the bovine growth hormone polyA sequence (bGHpA) were each obtained from the JW4303 vector construct (gift of Dr. Harriet Robinson, University of Massachusetts) and inserted into a plasmid backbone. The resultant construct was cut with NheI, filled with polymerase and then cut with BamHI to generate a vector fragment containing the pUC19 origin of replication, the ampicillin resistance gene and the bGHpA sequence. The plasmid backbone was cut a second time with SalI, filled with polymerase, and cut with BamHI to liberate a vector fragment containing the CMV-IA-TPA vector fragment. The two vector fragments were ligated together to yield a construct termed pWRG7054.

The pWRG7054 construct was cut with NheI, filled with polymerase, and cut with BamHI to produce a vector fragment. The HBcAg vector construct was cut with NcoI, filled with polymerase, and cut with BamHI to produce an insert fragment. The two fragments were then ligated together to yield a construct termed pWRG7063.

PEL-Bos was cut with EcoRI and dephosphorylated with calf intestinal phosphatase to produce a vector fragment. The pWRG7063 plasmid was cut with HindIII, filled with polymerase, and cut with EcoRI to produce an insert fragment containing the hTPA signal peptide, the HBcAg antigen sequence and the bGHpA region. These two fragments were ligated together to provide a construct termed pWkG7145.

The pWRG7128 construct was cut with EcoRI and dephosplhorylated with calf intestinal phosphatase to produce a vector fragment containing the HbsAg coding region under transcriptional control of the hCMV promoter. The pWRG7145 construct was cut with MfeI and EcoRI to produce an insert fragment comprised of the HELF promoter/intron, the hTPA signal peptide sequence, the HBcAG antigen sequence and the bGHpA region. These fragments were then ligated together to provide the pPJV7193 plasmid construct containing the HBcAg and HBsAg coding sequences.

B. Vaccine Preparation and Immunization

A panel of DNA vaccine compositions was assembled using various combinations of the following DNA plasmids: the pPJV7193 construct (encoding the hepatitis B surface antigen and hepatitis B core antigen); the pPJV7046 construct (a DNA plasmid vector containing the same CMV promoter/Intron A combination of pWRG7128 but encoding an irrelevant Beta-galactosidase antigen from S. thermophilus); and the pTRANCE construct (encoding the TRANCE cytokine). The final DNA concentration in each vaccine composition was; 2.0 μg DNA/mg gold, and the concentration of the antigen construct (pPJV7193) was kept constant in all compositions, while the concentration of the TRANCE construct was varied. The actual concentrations of each constituent present in the panel of DNA vaccine compositions are reported in Table 4 below.

TABLE 4
TRANCE DNA Vaccine Compositions

| | Concentration (μg DNA/mg gold) | | |
|---|---|---|---|
| Ratio (pPJV7193:pTRANCE) | pPJV7193 | pPJV7046 | pTRANCE |
| (control) | 1.0 | 1.0 | 0 |
| 1:1 | 1.0 | 0 | 1.0 |
| 5:1 | 1.0 | 0.8 | .2 |
| 25:1 | 1.0 | 0.96 | 0.04 |
| 125:1 | 1.0 | 0.992 | 0.008 |
| 625:1 | 1.0 | 0.9984 | 0.0018 |

Plasmid DNA (pPJV7193, pPJV7046 and pTRANCE) was combined at the ratios reported in Table 4 above and then the plasmid mixture was coated onto 1-3 μm gold particles using the technique described above in Example 1 to obtain a final concentration of 2 μg DNA/mg gold. The DNA-coated gold particles were then loaded into Tefzel® tubing as in Example 1 above, and cut into lengths to serve as cartridges in the PowderJect® XR particle delivery device. Each cartridge contained 0.5 mg gold particles.

Six experimental groups of 4 Balb/c mice each were assembled and immunized (primed) with the DNA vaccine compositions listed in Table 4 above by particle-mediated delivery of the plasmid DNA coated onto gold-particles. For immunization, mice were shaved and the particles delivered to the abdomen skin in a single shot using a particle delivery device operated at 500 psi of helium. The mice were sacrificed at two weeks post immunization, and the spleens were removed for ELISPOT analysis.

C. ELISPOT Analysis

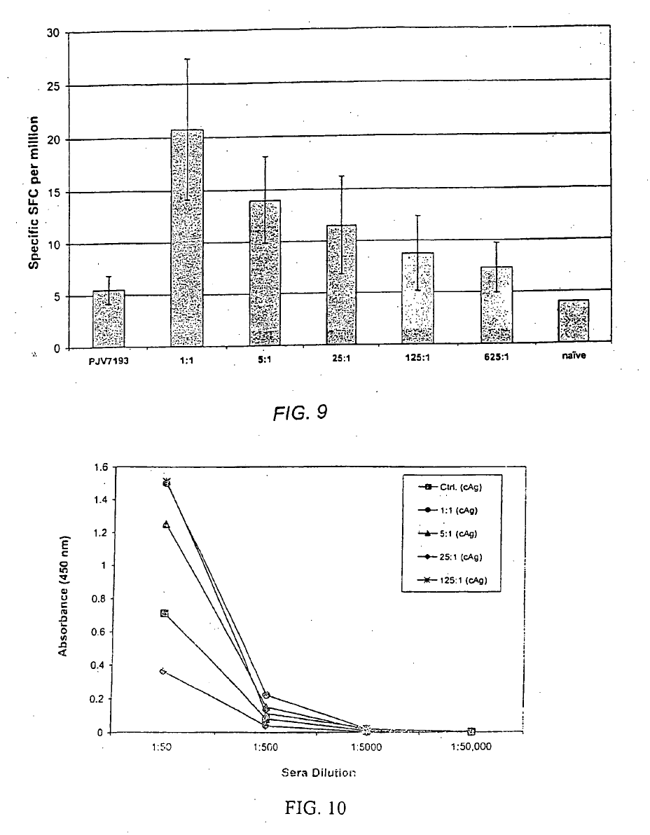

An ELISPOT filter plate was coated with rat anti-mouse IL-4 antibodies at a concentration of 0.75 μg/well in a 0.1M carbonate buffer. The plate was incubated overnight at 4° C. After washing two times with phosphate buffered saline (PBS), the plate was blocked with 100 μl of RPMI medium supplemented with 10% fetal bovine serum (FBS) for one hour at 37° C. The media was discarded after blocking. Splenocytes, with the red blood cells lysed and resuspended at 1×10⁷ cells/ml in RPMI (supplemented with 10% FBS, sodium pyruvate and non-essential amino acids) were added at a concentration of 1×10⁶ cells per well or 0.5×10⁶ cells per well. 100 μl of whole hepatits B virus core antigen (BioDesign), diluted to 20 μg/nil in RPMI supplemented with 10% FBS was added to the wells and the plate was incubated at 37° C. for 48 hours. After incubation, the cells and media were discarded and the plate washed two times with PBS, followed by a deionized water wash to lyse any remaining cells and then washed two more times with PBS. Detection antibody (biotinylated rat anti-mouse IL-4) was diluted to 1 μg/ml in PBS, and 50 μl was added to each well and incubated at room temperature for 1 hour. The plate was then washed five times with PBS, and 50 μl of strepavidin alkaline phosphatase conjugate (diluted 1:1000 in PBS) was added to each well. Following a 1 hour incubation at room temperature, the plate was washed five times with PBS, and 50 μl of a chromagenic alkaline phosphatase substrate was added to each well. As soon as spots emerged, the reaction was stopped by rinsing the plate with tap water. Spots were counted and the results were graphed as specific spots per million splenocytes in FIG. 9. As can be seen by review of the data depicted in FIG. 9, IL-4 production specific to the HBcAg was increased relative to the control (non-pTRANCE containing composition), and showed a dose-related decrease in specific spots.

D. Antibody Analysis (ELISA)

The above-described study was repeated with coated DNA particle cartridges prepared as before, but with the following modifications: the pPJV7193 construct (encoding the hepatitis B surface antigen and hepatitis B core antigen) and the pTRANCE construct (encoding the TRANCE cytokine) were coated onto separate batches of gold particles (the pTRANCE and pPJV7046 plasmids were combined as above and the combination coated onto gold particles, but the pTRANCE plasmid was coated onto a separate batch of gold particles). The two batches of coated gold particles were then mixed together prior to coating the Tefzel® tubing. The same concentrations of DNA as reported in Table 4 above were used with the one exception that the 625:1 ratio was not included in this second study. Experimental groups of 4 Balb/c mice each were immunized as before, and the mice were sacrificed at 2 weeks post immunization and serum collected for antibody analysis by ELISA.

For the antibody analysis, an ELISA plate was coated with 100 μl of whole hepatits B virus antigen (BioDesign); diluted to 0.1 μg/ml in PBS and incubated at 4° C. overnight. The plate was washed one time with PBS with 0.05% Tween 20 (PBS-T), then blocked with 300 μl of blocking solution (PBS plus 5% dried milk) for 1 hour at room temperature. The blocking solution was discarded and 100 μl of serially diluted serum was added. The plate was incubated for 1 hour at room temperature, followed by washing three times with PBS-T. A conjugated antibody (goat anti-mouse IgG-horse radish peroxidase) was diluted 1:5000 in PBS plus 2% dried milk. 100 μl of the conjugated antibody as added to each well, and the plate was incubated for 1 hour at room temperature. The plate was then washed five times with PBS-T and 100 μl of TMB substrate was added to each well. After 15 minutes, the reaction was stopped with 100 μl of 1N H₂SO₄. Absorbance was read at 450 nm. The geometric mean of each experimental group was then calculated and graphed against the dilution in FIG. 10. As can be seen by review of the data depicted in FIG. 10, there was an increased anti-HBcAg antibody response in all groups receiving the pTRANCE adjuvant composition except for the composition containing the 25:1 ratio.

Accordingly, addition of the pTRANCE cytokine encoding plasmid construct in the hepatitis DNA vaccine compositions enhanced both the cellular and humoral immune responses to the antigen of interest.

Accordingly, novel compositions for elicting an immune have been described. Methods of using these compositions have also been described. Although preferred embodiments of the subject invention have been described in some detail, it is understood that obvious variations can be made without departing from the spirit and the scope of the invention as defined by the appended claims.

1-41. (canceled)

42. A vaccine composition, comprising (a) at least one peptide antigen or an expression vector comprising a polynucleotide encoding at least one antigen; and (b) at least one cytokine, or an expression vector comprising a polynucleotide encoding at least one cytokine, where the cytokine is selected from the group consisting of CD40 ligand (CD40L), tumor-necrosis factor-related activation-induced cytokine (TRANCE) and Flt3 ligand (flt-3L).

43. The vaccine composition of claim 42, wherein both the antigen and the cytokine are encoded by an expression vector or expression vectors.

44. The vaccine composition of claim 43, wherein both the antigen and the cytokine are encoded by the same expression vector.

45. The vaccine composition of claim 43, wherein the antigen and the cytokine are encoded by separate expression vectors.

46. The vaccine composition of claim 42, wherein the expression vector or expression vectors are coated onto core carriers.

47. The vaccine composition of claim 46, wherein the core carriers comprise gold.

48. The vaccine composition of claim 42, wherein the antigen, or encoded antigen, is a cancer antigen, an antigen from a pathogen or an allergy antigen.

49. The vaccine composition of claim 48, wherein the antigen, or encoded antigen, is a viral antigen.

50. The composition of claim 49, wherein the antigen, or encoded antigen, is selected from a herpes simplex virus antigen, a hepatitis virus antigen, a papilloma virus antigen and an influenza virus antigen.

51. The vaccine composition of claim 42, wherein the encoded antigen is expressed from a promoter derived from a gene encoding a co-stimulatory molecule.

52. The vaccine composition of claim 51, wherein the co-stimulatory molecule is CD80 or CD86.

53. A method for eliciting an immune response in a vertebrate subject, said method comprising administering to the subject: (a) at least one peptide antigen or an expression vector comprising a polynucleotide encoding at least one antigen; and (b) at least one cytokine, or an expression vector comprising a polynucleotide encoding at least one cytokine, where the cytokine is selected from the group consisting of CD40 ligand (CD40L), tumor-necrosis factor-related activation-induced cytokine (TRANCE) and Flt3 ligand (flt-3L), whereby the antigen is administered in, or is expressed in, an amount sufficient to elicit an immune response.

54. The method of claim 53, wherein administration is repeated to provide a prime and a booster administration.

55. The method of claim 53, wherein both the antigen and the cytokine are encoded by an expression vector or expression vectors.

56. The method of claim 55, wherein both the antigen and the cytokine are encoded by the same expression vector.

57. The method of claim 55, wherein the antigen and the cytokine are encoded by separate expression vectors.

58. The method of claim 53, wherein the expression vector or expression vectors are coated onto core carriers.

59. The method of claim 58, wherein the core carriers comprise gold.

60. The method of claim 53, wherein the antigen, or encoded antigen, is a cancer antigen, an antigen from a pathogen or an allergy antigen.

61. The method of claim 53, wherein the antigen, or encoded antigen, is a viral antigen.

62. The method of claim 53, wherein the antigen, or encoded antigen, is selected from a herpes simplex virus antigen, a hepatitis virus antigen, a papilloma virus antigen and an influenza virus antigen.

63. The method of claim 53, wherein the encoded antigen is expressed from a promoter derived from a co-stimulatory molecule.

64. The method of claim 53, wherein the co-stimulatory molecule is CD80 or CD86.


SEQUENCE LISTING (8 sequences)

SEQ ID NO: 1 - DNA, 32 bp, Artificial Sequence
  Description of Artificial Sequencesynthetic construct
acgcgtcgactctagaaggagacattcagctg

SEQ ID NO: 2 - DNA, 32 bp, Artificial Sequence
  Description of Artificial Sequencesynthetic construct
acgcgtcgacagctttcatggcctagctgcta

SEQ ID NO: 3 - DNA, 34 bp, Artificial Sequence
  Description of Artificial Sequencesynthetic construct
attcggccgcggtctagagccaatggagcttagg

SEQ ID NO: 4 - DNA, 32 bp, Artificial Sequence
  Description of Artificial Sequencesynthetic construct
attcggccgcggagagttctgaatcagggtgt

SEQ ID NO: 5 - DNA, 29 bp, Artificial Sequence
  Description of Artificial Sequencesynthetic construct
acgcgtcgacagtcttcctcatcccacca

SEQ ID NO: 6 - DNA, 29 bp, Artificial Sequence
  Description of Artificial Sequencesynthetic construct
acgcgtcgaccatcacacagcaaggctag

SEQ ID NO: 7 - DNA, 29 bp, Artificial Sequence
  Description of Artificial Sequencesynthetic construct
acgcgtcgacgtttgttagtccatgcacg

SEQ ID NO: 8 - DNA, 25 bp, Artificial Sequence
  Description of Artificial Sequencesynthetic construct
tccccgcggagagaggcgacatttc